(19) United States
(12) Patent Application Publication
(10) Pub. No.: US 2012/0269842 A1
(43) Pub. Date: Oct. 25, 2012

(54) ENTEROTOXIGENIC E. COLI FUSION PROTEIN VACCINES

(75) Inventor: Weiping Zhang, Brookings, SD (US)

(73) Assignee: SOUTH DAKOTA STATE UNIVERSITY, Brookings, SD (US)

(21) Appl. No.: 13/501,023

(22) PCT Filed: Oct. 8, 2010
(86) PCT No.: PCT/US10/52041

§ 371 Date: Jul. 2, 2012

Related U.S. Application Data
(60) Provisional application No. 61/250,427, filed on Oct. 9, 2009.

(51) Int. Cl. A61K 39/108; A61P 37/04; C12N 15/31

(52) U.S. Cl. 424/190.1; 536/23.7

(57) ABSTRACT

The disclosure relates to fusions of enterotoxigenic Escherichia coli (ETEC) polynucleotides and polypeptides. In one example, a toxiod heat-labile (LT) and heat-stable (STa) fusion is described. This fusion may include LT with a substituted amino acid at position 192, a linker, and STa with a substituted amino acid at positions 11, 12, 13, or 14. Further provided are vaccines containing the disclosed fusions. Example methods for administering to a subject the disclosed vaccines and using the fusions and vaccines to reduce or eliminate contamination of a food or water supply are additionally contemplated.

### RELATED APPLICATIONS

This application claims priority under 35 U.S.C. §119(e) to U.S. Provisional Patent Application No. 61/250,427, filed Oct. 9, 2009, and PCT Patent Application No. PCT/US2010/52041, filed Oct. 8, 2010, both of which are hereby incorporated by reference in their entirety.

### STATEMENT REGARDING FEDERALLY SPONSORED RESEARCH OR DEVELOPMENT

This invention was made with U.S. Government support from the following agency: XXX. The U.S. Government has certain rights in this invention.

### TECHNICAL FIELD

The present disclosure relates to vaccines. More particularly, the embodiments of the present disclosure encompass fusion proteins of STa toxoid and LT toxoid, which can be used in vaccines against entertoxigenic Escherichia coli (ETEC). A method of making the vaccines is also contemplated.

### BACKGROUND

Escherichia coli (E. coli) are fairly ubiquitous bacteria. Many E. coli strains are harmless; however, ETEC is a major cause of illnesses, such as intestinal disease and/or diarrhea in man and farm animals. Of the total number of cases of worldwide diarrhea, 210 million are caused by ETEC, and 380,000 cases end in death each year. This E. coli strain is the principal causal agent of traveler's diarrhea. In farm animals, ETEC is equally as devastating. In the North American swine industry, neonatal and post weaning diarrhea caused by ETEC is one of the most economically important porcine diseases. For example, ETEC strains are believed to be responsible for the death of 10.8% of all pre-weaned pigs and up to more than 3% of all weaned pigs.

ETEC infection is generally acquired orally, principally through contaminated food or drink; the bacterium overcomes the acidic conditions of the stomach until it reaches the small intestine, where it adheres to the intestinal mucosa and liberates its two principal enterotoxins, heat-labile enterotoxin (LT) and heat-stable enterotoxin (ST). These two enterotoxins are principal factors responsible for ETEC related diarrhea.

Due to the epidemiological importance of ETEC, many efforts have been directed to the prevention of the illness by obtaining an effective and safe vaccine; however, to date, these efforts have been unsuccessful due to toxicity and immune response issues. Therefore, it is of great relevance to continue toward an effective way to treat ETEC related disease.

### SUMMARY

Disclosed are isolated polynucleotides and polypeptides encoded therein. The disclosed polynucleotides may include an isolated polynucleotide comprising a coding sequence for STa. In some embodiments, the disclosed STa polypeptides are toxoid forms of STa. The STa toxoids include non-native amino acid substitutions in some embodiments. Further, some embodiments include STa amino acid substitutions, which do not disrupt the disulfide bonds found in native STa. The STa toxoid may be operably connected to an LT polypeptide, such as in the case of a fusion protein. The LT polypeptide may also be a toxoid form of LT.

Host cell strains expressing the toxoids are also disclosed. In one embodiment, a host cell strain is an Escherichia coli strain which expresses an STa toxoid operably linked to an LT toxoid. The STa toxoid may have a non-native amino acid at amino acid 13 and the LT toxoid may have a non-native amino acid at amino acid 192.

The disclosed polynucleotides or polypeptides may be formulated as a pharmaceutically effective therapeutic that comprises the polynucleotides or polypeptides together with a pharmaceutical excipient. The pharmaceutically effective therapeutic may be a vaccine. The vaccine may be a live vaccine comprising a host cell capable of expressing the disclosed toxoids. The pharmaceutically effective therapeutics may be administered in a method for treating, preventing or reducing the effect of ETEC disease. In some embodiments, the pharmaceutically effective therapeutics comprise a fusion protein of an STa toxoid and an LT toxoid. The pharmaceutically effective therapeutics may be administered in a method to a subject in need thereof.

### BRIEF DESCRIPTION OF THE DRAWINGS

FIG. 1a demonstrates construction of a porcine ‘LT₁₉₂:STa_{-toxoid}’ genetic fusion polypeptide. PCR primers 184EcoRV-Fand LT-R amplified the entire porcine eltAB genes (without stop codon), and primers STa-F and pBREagI-R amplified the full-length porcine estA gene (without signal peptide). Primers LT₁₉₂-R and LT₁₉₂-F; complementary to LT₁₉₂-R, mutated eltAB genes for LT₁₉₂, and primers mSTa12-R and mSTa12-F; complementary to mSTa12-R, mutated the STa gene for an STa mutant. Primers pLT:STa-R and pLT:STa-F added a ‘Gly-Pro’ linker and genetically fused the mutated LT genes and the mutated STa gene. Gene sizes are not proportional. The inserted picture at the right lower corner shows Western blot detection of toxoid fusion using anti-CT and anti-STa antibodies, and total protein samples from TOPO cells were used as a negative control.

FIG. 1b demonstrates construction of human ‘LT₁₉₂:STa₁₃’ genetic fusions. Fusions 1 and 2 genes had one STa₁₃ gene genetically fused at the 3′ end of the LT₁₉₂ genes (3′ end of the eltB gene), with a ‘Gly-Pro’ linker in fusion 1 gene and an ‘L-linker’ in fusion 2 gene. Fusion 3 gene had one STa₁₃ gene fused at the 5′ end of the LT₁₉₂ genes with a ‘Gly-Pro’ linker. Fusion 4 gene had a single STa₁₃ gene inserted between the A1 and A2 fragments of the LT₁₉₂ genes by a ‘SalI-linker’, and fusion 5 gene had the STa₁₃ gene inserted at the end of the signal peptide of the eltB gene of the LT₁₉₂ genes with the ‘Gly-Pro’ linker.

FIG. 2 is an STa competitive ELISA showing detection of expression of porcine STa proteins among the STa recombinant 8330 (STa) and mutant strains 8413 (STa₁₁), 8415(STa₁₂) and 8417(STa₁₃). 1.25 ng STa-ovalbumin conjugate was coated on each well, and anti-STa serum (1:10,000) was used as the primary antibodies and horseradish peroxidase-conjugated goat anti-rabbit immunoglobulin (IgG; 1:10,000) was used as the secondary antibodies. Optical densities were measured at 405 nm.

FIG. 3 is a cyclic GMP ELISA detecting the toxicity of STa toxoids. Culture growth supernatant from STa toxoid strains was used to stimulate T-84 cells for an increase of intracellular cGMP levels by using cGMP EIA kit (Assay Design).

FIG. 4 is a porcine ligated gut loop assay to detect STa toxoid toxic activity. 2×10⁹ CFUs of 8330 (STa), 8413 (STa₁₁), 8415(STa₁₂), 8417(STa₁₃) or a negative control 8331(−) strain were incubated in each loop (three repeats). After an 8 hour incubation, fluid accumulated in each loop was measured, and a ratio of the accumulated fluid (gram) and the loop length (cm) was used as an index.

FIG. 5 demonstrates detection of fusion 1-5 proteins in GM1 ELISA and SDS-PAGE with anti-CT antiserum. Supernatant and pellet samples of fusion 1-5 strains, from equivalent amounts of bacterial cells (overnight culture growth), were used in GM1 ELISA. Rabbit anti-CT serum (1:3300) and HRP-conjugated goat anti-rabbit IgG (Sigma, 1:5000) were used as the primary and secondary antibodies. Optical densities were measured at 405 nm after 20 min reaction. Boxes and error bars were means and standard deviations. The p values in black color were resulted from comparison of supernatant samples between each fusion strain and the negative control strain (1836-2), and the p value in grey color were resulted from comparison of pellet protein samples. The inserted image was results from Western blot. Total proteins prepared from bacterial culture pellets of fusion 1 strain (lane 1), fusion 2 strain (lane 2), fusion 3 strain (lane 3), 1836-2 (lane 4), fusion 4 strain (lane 5) and fusion 5 strain (lane 6), the same used in GM1 ELISA, were separated by 13% SDS PAGE gel; rabbit anti-CT serum (1:3300; Sigma) was used as the primary antibodies and HRP-conjugated goat anti-rabbit IgG (1:5000; Sigma) as the secondary antibodies. The SuperSignal West Pico chemiluminescent substrate kit (Pierce) was used for detection.

FIG. 6a is antibody titration from serum and fecal samples of rabbits immunized with ‘pLT₁₉₂:pSTa₁₂’ or ‘pLT₁₉₂:pSTa₁₃’ fusion antigenic polypeptides. The titers (in log 10) of anti-LT was detected in an LT GM1 ELISA using purified CT and antigen, and rabbit antiserum samples (1:50) as the primary antibody. To titer anti-STa antibody, STa ovalbumin-conjugates were used as an antigen, and rabbit antiserum and antifecal samples (1:50) as the primary antibody. HRP-conjugated IgG and IgA antibodies (1:5000) were used as the secondary antibodies. Optical densities greater than 0.4 (after subtracting the background reading) were used to calculate antibody titers (in log 10).

FIG. 6b shows titration of anti-STa antibodies in serum and fecal samples of mice immunized with purified 6×His-tagged human fusion antigenic polypeptide. 1.25 ng of ovalbumin-STa conjugates were coated in each well, and 200 μl of serum (1:50) or fecal (1:50) samples from each mouse immunized with purified 6×His-tagged fusion 1b, fusion 2b, fusion 3b, fusion 4b, fusion 5b, or the control group (in triplicates) was added to the wells in the first row (in triplicates) and subsequently in binary dilution. HRP-conjugated goat anti-mouse IgG and IgA (1:3300) were used as the secondary antibodies, respectively. Optical densities of greater than 0.4 (after subtracting the background reading) were used to calculate antibody titers (in log 10). Boxes and error bars indicate means and standard deviations.

FIG. 6c illustrates titration of anti-LT antibodies in mouse serum and fecal samples in GM1 ELISA. 40 ng GM1 (Sigma) and 200 ng CT (Sigma) were used in GM1 ELISA. 200 of serum (1:50) or fecal (1:50) samples from each mouse immunized with purified human 6×His-tagged fusion 1b, fusion 2b, fusion 3b, fusion 4b, fusion 5b, or the control group (in triplicates) were added to the wells of the first row and subsequently in binary dilution (in triplicates). HRP-conjugated goat anti-mouse IgG and IgA (1:3300) were used as the secondary antibodies, respectively. Optical densities of greater than 0.4 (after subtracting the background reading) were used to calculate antibody titers (in log 10). Boxes and error bars indicate means and standard deviations.

FIG. 7a demonstrates anti-LT and anti-STa antibody neutralization. Serum and fecal samples (1:50) from rabbits immunized with porcine ‘pLT₁₉₂:pSTa₁₂’ or ‘pLT₁₉₂:pSTa’ fusion antigenic polypeptide were used to neutralize purified CT (10 ng) or STa (2 ng). The mixture was added to T-84 cells to test any increasing of intracellular cGMP (STa; Assay Design) or cAMP (CT; Invitrogen) levels. Cell culture medium alone was included as a negative control. CT or STa toxin alone, or incubated with a serum or a fecal sample from the control rabbit, were included as the negative control.

FIG. 7b demonstrates anti-STa antibody neutralization against STa toxin in T84 cells. Serum and fecal samples (1:5) from mice immunized with human 6×His-tagged fusion 1-5b were used to neutralize STa toxin. 150 μl serum or fecal samples (1:5 dilution; in triplicates) from each group of mice was mixed with 2 ng STa toxin (in 150 μl of DMEM/F12 medium) and incubated at room temperature for 1 h. 150 μl of each mixture was added to a well containing T-84 cells (1×10⁵ per well) to test intracellular cGMP levels with an ELISA kit (EIA kit, Assay Design). Serum and fecal from the control group was included and treated the same as other samples. Intracellular cGMP concentration (pmol/ml) was calculated by following the manufacturer's protocol. P values inside each box were calculated by comparing to the negative control in student t-test. Boxes and error bars indicate means and standard deviations.

FIG. 8 shows ELISA detection of anti-STa IgA antibodies in colostrum samples of a sow immunized with porcine ‘LT₁₉₂:STa₁₂’ antigenic polypeptide, a negative control sow, and serum samples of the born piglets. Each well was coated with 1.25 ng STa-ovalbumin and then 100 μl of each sow colostrum sample (1:10) and piglet serum sample (1:50) was added. HRP-conjugated goat-anti-porcine IgA (1:3000) was used as the secondary antibody. Optical densities were measured at 405 nm.

### DETAILED DESCRIPTION

In describing the invention herein, include exemplary embodiments, it is to be understood that the embodiments are not limited to particular compositions or methods, as the compositions and methods can, of course, vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which a claim pertains. Many methods and compositions similar, modified, or equivalent to those described herein can be used in the practice of the current embodiments without undue experimentation.

As used in this specification and the appended claims, the singular forms “a,” “an” and “the” can include plural referents unless the content clearly indicates otherwise. Thus, for example, reference to “a polypeptide” can include a combination of two or more polypeptides. The term “or” is generally employed to include “and/or,” unless the content clearly dictates otherwise.

As used herein, “about,” “approximately,” “substantially,” and “significantly” will be understood by person of ordinary skill in the art and will vary in some extent depending on the context in which they are used. If there are uses of the term which are not clear to persons of ordinary skill in the art given the context in which it is used, “about” and “approximately” will mean plus or minus ≦10% of particular term and “substantially” and “significantly” will mean plus or minus >10% of the particular term.

Units, prefixes, and symbols may be denoted in their SI accepted form. Numeric ranges recited within the specification are inclusive of the numbers defining the range and include each integer within the defined range.

### I. STa/LT Chimeras

“ST” is understood to refer to the heat-stable enterotoxin produced by ETEC. ST is of a low molecular size (approximately 4000 daltons) and resistant to boiling for 30 minutes. There are several variants of ST, of which STa or STp is found in isolates from both human and non-human subjects whereas STb or STh is predominantly in human isolates. STa is known to act by binding to guanylate cyclase that is located on the apical membranes of host cells. Once the enzyme is bound, it is activated, which leads to secretion of fluid and electrolytes. Generally, STa becomes immunogenic only if coupled to a strongly immunogenic carrier protein. However, even in situations where STa is coupled to a strongly immunogenic carrier protein, it may retain toxicity or stay poorly immunogenic. Sears, C. L. and Kaper, J. B. 1996. “Enteric Bacterial Toxins: Mechanisms of Action and Linkage to Intestinal Secretion.” Microbiol. Rev. 60: 167-215. Generally the STa polynucleotide will be only the portion of the STa polynucleotide that encodes the mature version of the polypeptide. When referring to an STa polypeptide herein, unless otherwise noted, STa is the mature or active version of the polypeptide, i.e., the polypeptide capable of binding to enzyme.

“LT” is understood to refer to the heat-labile enterotoxin produced by ETEC. LT is similar in molecular size, sequence, antigencity, and function to the cholera toxin. In isolates from humans, it is an 86 kD protein composed of an enzymatically active (A) subunit surrounded by 5 identical binding (B) subunits. It binds to ganglioside receptors that are also recognized by cholera toxin and its enzymatic activity is similar to that of cholera toxin. Sears, C. L. and Kaper, J. B. 1996. “Enteric Bacterial Toxins: Mechanisms of Action and Linkage to Intestinal Secretion.” Microbiol. Rev. 60: 167-215.

“Nucleotide” refers to a phosphate ester of a nucleoside, as a monomer unit or within a nucleic acid. Nucleotides are sometimes denoted as “NTP” or “dNTP” and “ddNTP” to particularly point out the structural features of the ribose sugar. The term “nucleic acid” encompasses the terms “oligonucleotide” and “polynucleotide” and means single-stranded or double-stranded polymers of nucleotide monomers, including 2′-deoxyribonucleotides (DNA) and ribonucleotides (RNA). The nucleic acid can be composed entirely of deoxyribonucleotides, entirely of ribonucleotides, or chimeric mixtures thereof, linked by internucleotide phosphodiester bond linkages, and associated counter-ions. The term also refers to nucleic acids containing modified bases.

The term “polypeptide” as used herein, refers to polymers of amino acids linked by peptide bonds and includes proteins, enzymes, peptides, and other gene products encoded by nucleic acids described herein. A “toxoid” is a toxin that has a decreased toxic effect but that retains its antigenic properties. Toxoids as used in the present disclosure are variant polypeptides.

“Native” proteins or polypeptides refer to wild-type proteins, or proteins or polypeptides with sequences identical to those of wild-type proteins and fragments thereof. A “native” polynucleotide is a wild-type nucleotide, or a nucleotide with a nucleotide sequence identical to a gene or fragment thereof found in a wild-type gene. “Recombinant” polypeptides refer to polypeptides produced by recombinant DNA techniques; i.e., produced from cells transformed by an exogenous DNA construct encoding the desired polypeptide. Unless modified by “variant,” “toxoid” or “mutation,” the recombinant polynucleotides and polypeptides disclosed herein have the same nucleotide or amino acid sequence as is found in the wild-type polynucleotide or polypeptide, and thus fall into the above definition of native. “Synthetic” polypeptides are those prepared by chemical synthesis.

As used herein, a “variant” or “mutant” refers to a polypeptide or a polynucleotide molecule having an amino acid sequence or nucleic acid sequence, respectively, that differs from a reference polypeptide or polynucleotide molecule, respectively. A variant or mutant may have one or more insertions, deletions or substitutions of an amino acid residue or nucleotide residue relative to a reference molecule. For example, a variant STa polypeptide may include hybrids or fusion polypeptides.

Variants, mutants, or hybrids (e.g., a variant STa or mutant polypeptide) may have 99%, 98%, 97%, 96%, 95%, 94%, 93%, 92%, 91%, 90%, 89%, 88%, 87%, 86%, 85%, 84%, 83%, 82%, 81%, 80%, 70%, 60% or 50% amino acid sequence identity (or nucleic acid sequence identity) relative to a reference molecule (e.g., relative to the native STa polypeptide or an STa/LT hybrid polypeptide). “Percentage sequence identity” may be determined by aligning two sequences using the Basic Local Alignment Search Tool (BLAST) available at the NBCI website.

Mutation of the disclosed polypeptides may be accomplished using any method known in the art. Mutation may be accomplished by mutating the gene that encodes STa or LT. In exemplary embodiments, mutation of the genes encoding the STa or LT polypeptides is done through site directed mutagenesis. In the current disclosure, mutation of polynucleotides will generally result in substitution of amino acids in the relevant polypeptide. However, mutation at the polynucleotide level, wherein the polynucleotide continues to encode the same amino acid is also contemplated. A polynucleotide mutation required to produce a particular amino acid is well understood by one of skill in the art.

One embodiment encompasses synthetic or recombinant STa with mutations at the 11^{th}, 12^{th} and/or 13^{th} amino acids of the STa polypeptide. In one embodiment, the STa estA gene from a porcine E. coli strain will be mutated. In other embodiments, the estA gene from a human E. coli strain will be mutated. When dealing with estA genes from human E. coli strains, amino acids will generally be mutated at the 12^{th}, 13^{th} and 14^{th} positions of the polypeptide. It is contemplated that mutation may take place at one or more of these amino acid positions. In certain cases, the mutation at these amino acid positions will be such that the overall three-dimensional shape of the STa protein is retained. For example, disulfide bonds present in the native STa protein will be retained. In some embodiments, immunogenicity of STa protein is increased as compared to native protein. In some embodiments, an epitope of STa protein is retained. In other embodiments, a new epitope is created.

In one embodiment, the disclosed polypeptide will have mutations at three amino acid positions as compared to the native human E. coli strain STa protein (SEQ ID NO: 1). In this embodiment, the disclosed polypeptide will have approximately 85% sequence identity to SEQ ID NO: 1, i.e. 3 amino acids out of 19. In another embodiment, the disclosed polypeptide will have mutations at only two amino acid positions are compared to the native human E. coli strain STa protein. In this embodiment, the disclosed polypeptide will have approximately 89% sequence identity to SEQ ID NO: 1, i.e. 2 amino acids out of 19. Variant polynucleotides of the native human E. coli strain STa nucleotide sequence (SEQ ID NO: 2) may encode the variant polypeptides.

In porcine embodiments, the disclosed polypeptide may have mutations at three amino acid positions as compared to the native porcine E. coli strain STa protein (SEQ ID NO: 3). In this embodiment, the disclosed polypeptide will have approximately 84% sequence identity to SEQ ID NO: 3, i.e. 3 amino acids out of 18. In another embodiment, the disclosed polypeptide has mutations at only two amino acid positions compared to the native porcine strain STa protein. In this embodiment, the disclosed polypeptide will have approximately 89% sequence identity to SEQ ID NO: 3, i.e. 2 amino acids out of 18. Variant polynucleotides of the native porcine E. coli strain STa nucleotide sequence (SEQ ID NO: 4) may encode the variant polypeptides.

In an exemplary embodiment, the native asparagine at amino acid 11 in the porcine E. coli strain protein (or the asparagine at amino acid 12 in the human E. coli strain protein) of the STa polypeptide will be replaced with lysine, arginine or glutamate. The native proline at amino acid 12 in the porcine E. coli strain protein (or the proline at amino acid 13 in the human strain protein) will be replaced with phenylalanine, glutamate or arginine in another embodiment. Alanine at amino acid 13 in the porcine E. coli strain protein (or the alanine at amino acid 14 in the human strain protein) may be replaced with glutamine. In addition to these specific mutations, polypeptides may be mutated by conservatively substituting amino acids with other amino acids. In some instances, the only requirement for substitution is that the non-native amino acid does not disrupt the formation of disulfide bonds which are found in the native protein.

LT may also be mutated. In one embodiment, the native LT protein (SEQ ID NO: 6) has at least one amino acid substitution. To mutate LT, the native arginine at amino acid 192 of the eltAB gene, can be changed to glycine. Generally in the currently disclosed embodiments, similarly to a mutation of STa, a mutation of LT will result in an LT protein with reduced toxicity. This reduced toxicity can be as compared to native protein. Mutation of the eltAB gene (SEQ ID NO: 5) has been described in U.S. patent application Ser. No. 12/169,259, herein incorporated by reference in its entirety.

Mutated STa can be fused to native, recombinant, or mutated LT to form a chimeric polynucleotide or polypeptide. “Fusion” and “chimera” are used interchangeably herein. STa and LT may be fused either directly or through a linker. A linker may include any stretch of polynucleotide or polypeptide, which allows maintenance of a particular STa/LT chimeric's function. In some embodiments, the chimeric's function will be as a non-toxic immunogen. In one embodiment, the linker will be a glycine-proline-glycine-proline polypeptide linker. The polypeptide linker may be constructed by a polynucleotide which encodes the linker, wherein the polynucleotide has a nucleotide sequence of gggccggggccc (SEQ ID NO: 7). In another embodiment, the linker is an L-linker with a nucleotide sequence of cgagctcggtacccggggatc (SEQ ID NO: 8).

In many embodiments, an STa/LT fusion protein will be constructed through fusion of estA and eltAB genes. These chimeric polynucleotides, made from any combination of mutated or recombinant genes encoding STa and LT are then translated into a fusion protein. For example, STa polypeptide mutated at position 11 (or 12) may be fused with LT polypeptide mutated at amino acid 192. In another embodiment, STa polypeptide mutated at amino acid 12 (or 13) may be fused with LT polypeptide mutated at amino acid 192. In yet another example, STa polypeptide mutated at amino acid 13 (or 14) may be fused with LT polypeptide mutated at amino acid 192. In one embodiment, the disclosed fusion protein comprises SEQ ID NO: 9.

In different embodiments, estA and eltAB genes are genetically fused at different positions. For example, in one embodiment, STa is fused at the 3′ end of the LT gene, i.e., STa₁₃ estA gene is fused to the 3′ end of the eltB gene. In another embodiment, STa is fused to the 5′ end of the LT gene, i.e., STa₁₃ gene is fused to the 5′ end of the eltB gene. In yet another embodiment, the STa gene is fused between different fragments of the LT gene, i.e., STa₁₃ is inserted between the A1 and A2 fragments of the eltA gene or fused to the 3′ end of the eltA gene. Furthermore, in certain fusions, the nucleotides coding trans-membrane signal peptides are removed. Different fusion positions may incorporate any of the linkers.

Generally, the fusion proteins will be formed by methods well understood in the art. For example, fusion proteins may be constructed by inserting a chimeric polynucleotide into an appropriate expression vector and then transforming host cells with the vector. Cells capable of expressing the disclosed polypeptides are known as host cells. Applicable vectors are not meant to be limiting, nor are applicable host cells. In one embodiment, the host cells will be non-pathogenic E. coli.

Host cells can be procaryotic and eukaryotic cells, either stably or transiently transformed, transfected, or electroporated with polynucleotide sequences in a manner which permits expression of STa and LT polypeptides. Expression systems of the invention include bacterial, yeast, fungal, viral, invertebrate, and mammalian cells systems. Host cells of the invention are a valuable source of immunogen for development of antibodies specifically immunoreactive with ETEC toxins. Furthermore, host cells are useful in methods for large scale production of ETEC antigenic polypeptides, wherein the cells are grown in a suitable culture medium and the desired polypeptide products are isolated from the cells or from the medium in which the cells are grown by, for example, immunoaffinity purification or any of the multitude of purification techniques well known and routinely practiced in the art. Any suitable host cell may be used for expression of the polypeptide, such as E. coli, other bacteria, including P. multocida, Bacillus and S. aureus, Lactobacillus or Salmonella strains, yeast, including Pichia pastoris and Saccharomyces cerevisiae, insect cells, or mammalian cells, including CHO cells, utilizing suitable vectors known in the art. In many embodiments, the host cells will be non-pathogenic E. coli, or Lactobacillus strains or Salmonella vaccine strains. Proteins may be produced directly or fused to a peptide or polypeptide, and either intracellularly or extracellularly by secretion into the periplasmic space of a host cell or into the cell culture medium.

### II. Vaccines

In some embodiments, the polynucleotides and polypeptides, including the chimeras, may be formulated as pharmaceutical compositions that include a therapeutically effective amount of the compounds. The pharmaceutical compositions may also include one or more pharmaceutically acceptable carriers. In some embodiments, pharmaceutically acceptable carriers are host cells. The polynucleotides and polypeptides disclosed herein may be administered to patients in need thereof. A “patient in need thereof” may include a patient that already has an ETEC infection or a patient at risk for contracting an ETEC infection.

For example, the polynucleotides and polypeptides may be administered in a therapeutically effective amount as a vaccine to treat or prevent ETEC infection. The vaccine response need not provide complete protection and/or treatment against ETEC infection or against colonization and shedding of ETEC. Even partial protection against colonization and shedding of ETEC bacteria will find use herein. “Colonization” refers to the presence of ETEC in the intestinal tract of a subject, such as a human. “Shedding” refers to the presence of ETEC in feces.

As used herein, a “patient” is interchangeable with “subject” and means an animal, which may be a human or non-human animal, in need of treatment. Non-human animals may include pigs, cows, horses, sheep, dogs, cats and the like. Humans specifically include children, including infants less than 1 year of age. Children may also be less than 5 years of age. In some embodiments, humans are adults. Although not meant to be limiting, these adults may be either international travelers, or military personnel deployed in areas with endemic ETEC infection.

The phrase “therapeutically effective amount” shall mean that the dosage of an active agent that provides the specific pharmacological response for which the active agent is administered in a significant number of subjects in need. A therapeutically effective amount of an active agent that is administered to a particular subject in a particular instance will not always be effective in treating or preventing the conditions/diseases described herein, even though such dosage is deemed to be a therapeutically effective amount by those of skill in the art.

A “vaccine” is a preparation of an attenuated or killed pathogen, such as a bacterium or virus, or of a portion of the pathogen's structure that upon administration stimulates antibody production or cellular immunity against the pathogen, but is incapable of causing severe infection.

A specific pharmacological response for a vaccine can be an immunological response. An “immunological response” to a composition or vaccine is the development in the subject of a cellular and/or antibody-mediated immune response to the composition or vaccine of interest. Usually, an immunological response includes, but is not limited to, one or more of the following effects: the production of antibodies, B cells, helper T cells, suppressor T cells, and/or cytotoxic T cells and or γδ T cells, directed specifically to an antigenic polypeptide or antigenic polypeptides included in the composition or vaccine of interest. The subject generally displays either a therapeutic or protective immunological response such that ETEC disease is lessened and/or prevented; resistance of the intestine to colonization with ETEC is imparted; the number of subjects shedding ETEC is reduced, the amount of ETEC shed by a subject is reduced, and/or the time period of ETEC shedding by a subject is reduced.

The term “immunogenic” refers to a polypeptide which elicits an immunological response as described above. “Antigen”, “antigenic” and “immunogen” are also included in this definition. An immunogenic polypeptide as used herein, includes the full-length sequence of the particular ETEC polypeptides in question, analogs thereof, aggregates, or immunogenic fragments thereof. An “immunogenic fragment” is a fragment of an ETEC polypeptide, which includes one or more epitopes and thus elicits an immunological response. Such fragments can be identified using any number of epitope mapping techniques well known in the art. The term “epitope” refers to the site on an antigen or hapten to which specific B cells and/or T cells respond. The term is used interchangeably with “antigenic determinant” or “antigenic determinate site.”

The amount of antigen in each vaccine dose is selected as an amount which induces an immunological response without significant, adverse side effects, such as is the case in typical vaccines. Such amount will vary depending on which specific immunogen is employed and how it is presented.

Generally it is expected that each human dose will comprise 0.1-1000 μg of antigen, 0.1-500 μg of antigen, 0.1-100 μg of antigen and 0.1-50 μg of antigen. For other species, the appropriate dose can be determined by one of skill in the art. An optimal amount for a particular vaccine can be ascertained by standard studies involving observation of appropriate immune responses in vaccinated subjects. Following an initial vaccination, subjects may receive one or more booster immunizations. The skilled artisan understands the appropriate spacing and dosage of any booster immunizations.

The amount of antigen in an individual vaccine dose will depend on the type of vaccine. Attenuated, toxoid, DNA, and conjugate vaccines, as well as both whole-agent and subunit vaccines are contemplated. Plotkin 2005. Vaccines: past, present and future. Nat. Med. 11(4): S5 provides a good review of the state of the art of vaccine types. Actual methods of preparing such dosage forms are known, or will be apparent, to those skilled in the art. See, e.g., Remington's Pharmaceutical Sciences, Mack Publishing Company, Easton, Pa., 18th edition, 1990.

Vaccine administration is not meant to be limiting. Routes of administration include, but are not limited to, oral, topical, subcutaneous, intramuscular, intravenous, subcutaneous, intradermal, transdermal and subdermal. Similarly to the dose amount, the type of administration will depend at least partially on the type of vaccine. Transcutaneous administration of E. coli vaccines is described in detail in U.S. Pat. No. 7,527,802. Vaccine can be administered in a single dose treatment or in multiple dose treatments (boosts) on a schedule and over a time period appropriate to the age, weight and condition of the subject, the particular vaccine formulation used, and the route of administration. In certain embodiments, a vaccine may be delivered orally, such as through inclusion in formula, milk, water or food. In certain embodiments, entire food and or water supplies will be treated with the vaccine.

In exemplary embodiments, vaccination will be used to improve food safety. For example, in one embodiment a subject such as a meat animal will be vaccinated to prevent accidental contamination of meat products or vaccinated to reduce frequency of contamination of meat products during processing of meat. In other embodiments, vaccine will be added to raw food stuff, such as lettuce or other vegetables in order to prevent infection of the subject eating the raw food stuff. In some embodiments, vaccine will be added to a food or water supply to prevent or reduce the chance of the spread of ETEC. Treated water and/or food supplies for ranches, farms, villages, towns, and cities are contemplated. In certain embodiments, the entire water/food supply will be treated, whereas in other embodiments, only parts of the water/food supply will be treated. As is understood by the skilled artisan, in many embodiments where food and water supplies are treated with vaccine, the vaccine will be a live vaccine.

Polynucleotides or polypeptides in vaccines may be administered alone, or mixed with a pharmaceutically acceptable carrier, vehicle or excipient. Suitable vehicles are, for example, water, saline, dextrose, glycerol, ethanol, or the like, and combinations thereof. In addition, the vehicle may contain minor amounts of auxiliary substances such as wetting or emulsifying agents, pH buffering agents, or adjuvants in the case of vaccine compositions, which enhance the effectiveness of the vaccine. In addition, an example carrier includes host cells containing the disclosed polynucleotides, in a form wherein the disclosed polypeptides can be produced. Suitable adjuvants are described further below. The compositions of the present invention can also include ancillary substances, such as pharmacological agents, cytokines, or other biological response modifiers.

As explained above, vaccine compositions of the present invention may include adjuvants to further increase the immunogenicity of one or more of the ETEC antigens. Such adjuvants include any compound or compounds that act to increase an immune response to an ETEC antigen or combination of antigens, thus reducing the quantity of antigen necessary in the vaccine, and/or the frequency of injection necessary in order to generate an adequate immune response. In certain instances, LT will be used as both an antigen and an adjuvant. Nevertheless, additional adjuvants are contemplated. Adjuvants may include for example, emulsifiers, muramyl dipeptides, pyridine, aqueous adjuvants such as aluminum hydroxide, chitosan-based adjuvants, and any of the various saponins, oils, and other substances known in the art, such as Amphigen, LPS, bacterial cell wall extracts, bacterial DNA, synthetic oligonucleotides and combinations thereof. For example, compounds which may serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrimethylammonium bromide, while synthetic nonionic agents are exemplified by glyceryl esters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oil, or oil-in-water emulsions in which the oil component is mineral oil, are examples. In this regard, a “mineral oil” is defined herein as a mixture of liquid hydrocarbons obtained from petrolatum via a distillation technique; the term is synonymous with “liquid paraffin,” “liquid petrolatum” and “white mineral oil.” The term is also intended to include “light mineral oil,” i.e., an oil which is similarly obtained by distillation of petrolatum, but which has a slightly lower specific gravity than white mineral oil. Suitable animal oils include, for example, cod liver oil, halibut oil, menhaden oil, orange roughy oil and shark liver oil, all of which are available commercially. Suitable vegetable oils include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include amines, quaternary ammonium compounds, guanidines, benzamidines and thiouroniums. Specific compounds include dimethyldioctadecylammonium bromide (DDA) and N,N-dioctadecyl-N,N-bis(2-hydroxy-yethyl)propanediamine (“pyridine”). Avridine is also a well-known adjuvant.

A vaccine may include the fusion proteins as the antigen or a vaccine may be live host cell such as E. coli transformed to produce an STa/LT fusion protein as an antigen. In certain embodiments, the vaccine will be composed of a strain of live, orally applicable E. coli that have been transformed with an STa/LT fusion protein. Live, transformed E. coli vaccines are known in the art. See U.S. Pat. No. 7,163,820. In some embodiments, more than one strain of transformed live E. coli will be used in the vaccine. For example, one strain of E. coli may express the STa mutant protein while another strain may express LT mutant protein and both strains may be present in a vaccine.

A method of producing an ETEC vaccine is also contemplated. For example, following construction of variant STa polynucleotides and variant LT polynucleotides, the variant polynucleotides can then be operably connected in a vector. The vector will generally be an expression vector. An appropriate host cell can be transformed with the vector. In one embodiment, the host cell is given directly to a subject as a vaccine. In another embodiment, the transformed host cell is amplified and the variant fusion protein is isolated. All or part of the variant fusion protein is then used as a vaccine component. Additional vaccine components, such as excipients and adujvants are also contemplated.

### EXAMPLES

The invention may be further clarified by reference to the following examples, which serve to exemplify some of the embodiments and not to limit the invention in any way. The experiments were performed using the methodology described below.

### I. Bacterial Strains and Plasmids

A. Porcine

A porcine E. coli field isolate G58-2 was used to construct experimental strains. Two plasmids expressing 987P fimbriae, pDMS167 and pDMS158 were used to express 987P fimbria in G58-2 and porcine STa constructs. Plasmid pACYC184 was used to clone and express the recombinant, mutated porcine STa gene and the LT and STa chimeric genes; whereas a TOPO TA cloning vector was used for fusion protein expression and purification. Plasmid pUC19 was also used to clone the STa gene for producing an STa challenge strain that had a higher toxin expression. All constructs were cultured in LB medium supplemented with chloramphenicol (20 μg/ml) or ampicillin (50 μg/ml).

Twelve strains including an STa recombinant (8330), 3 STa mutants (8413, 8415, 8417), 4 LT and STa toxoid fusion strains (8474, 8475, 8552, 8554), 2 host strains (8227, 8795), a negative control (8331), and an STa challenge strain (8823) were constructed (Table 1a). After confirming the expression of STa toxoid proteins and assessing the toxicity and biological activity of each toxoid, STa₁₂ and STa₁₃ toxoids were selected for construction of toxoid fusions. Both resultant toxoid fusions were used to immunize adult rabbits, and the ‘LT₁₉₂:STa₁₃’ fusion was used to immunize a pregnant sow. Elicited anti-LT and anti-STa antibodies were titrated and examined for activity in neutralizing CT and STa toxins, and anti-STa antibodies were tested preliminarily in protection against an STa producing ETEC strain.

TABLE 1a
Escherichia coli strains and plasmids used in the study.
A porcine E. coli field isolate G58-2 was used as a parental strain and was
transformed with a plasmid pDMS167 or pDMA158 to express
987P fimbriae (G58/987P). This G58/987P was further transformed with
plasmids expressing a native pSTa, a mutated pSTa, or a
‘pLT₁₉₂:STa-_{toxoid}’ chimeric plasmid for a porcine STa
recombinant strain, three STa mutant strains, and
‘pLT₁₉₂:pSTa₁₂’ and ‘pLT₁₉₂:pSTa₁₃’ fusion constructs. In addition,
TOPO 10 cells and TA cloning vectors (Invitrogen) were used for
expression and purification of the ‘pLT₁₉₂:pSTa₁₂’ and
‘pLT₁₉₂:pSTa₁₃’ fusion proteins.

| Strains | Relevant properties | Plasmid |
|---|---|---|
| G58-2 | non-pathogenic porcine E. coli field isolate | |
| 04-21018 | a porcine ETEC isolate | (F18/STa/STb/ |
| | | Stx2e) |
| 8221 | LT₁₉₂ construct, 1836-2/pLT₁₉₂ | pLT₁₉₂/ |
| | | pBR322 |
| 8227 | a host strain, G58-2/987P | pDMS167 |
| 8795 | a host strain, G58-2/987P | pDMS158 |
| 8331 | a control, G58-2/987P/pACYC184 | pACYC184 |
| 8330 | pSTa recombinant, G58-2/987P/STa | pSTa/ |
| | | pACYC184 |
| 8413 | pSTa₁₁ mutant, G58-2/987P/STa₁₁ | pSTa₁₁/ |
| | | pACYC184 |
| 8415 | pSTa₁₂ mutant, G58-2/987P/STa₁₂ | pSTa₁₂/ |
| | | pACYC184 |
| 8417 | pSTa₁₃ mutant, G58-2/987P/STa₁₃ | pSTa₁₃/ |
| | | pACYC184 |
| 8474 | pLT₁₉₂:pSTa₁₂, G58-2/987P/LT₁₉₂:PSTa₁₂ | pLT₁₉₂:pSTa₁₂/ |
| | | pACYC184 |
| 8475 | pLT₁₉₂:pSTa₁₃, G58-2/987P/LT₁₉₂:pSTa₁₃ | pLT₁₉₂:pSTa₁₃/ |
| | | pACYC184 |
| 8552 | pLT₁₉₂:pSTa₁₂, TOPO 10/LT₁₉₂:STa₁₂ | pLT₁₉₂:pSTa₁₂/ |
| | | TA vector |
| 8554 | pLT₁₉₂:pSTa₁₃, TOPO 10/LT₁₉₂:STa₁₃ | pLT₁₉₂:pSTa₁₃/ |
| | | TA vector |
| 8823 | pSTa challenge strain, G58-2/987P/pSTa | pSTa/pUC19 |

B. Human

ETEC prototype strain H10407 was used to isolate the eltAB genes (coding LTAB) and estA gene (coding STa). E. coli BL21 (GE Healthcare, Piscataway, N.J.) and 1836-2 were used as parent strains. Vectors pBR322 (Promega, Madison, Wis.) and pET28a (Invitrogen, Carlsbad, Calif.) were used for cloning and expression of the LT, LT₁₉₂, STa, STa₁₃, and LT₁₉₂-STa₁₃ genes. All E. coli strains were cultured in LB medium supplemented with kanamycin (30 μg/ml) or ampicillin (50 μg/ml).

TABLE 1b
Escherichia coli strains and plasmids used in the study.
E. coli strain BL21 and a porcine field isolate 1836-2 were used as parent strains to
express ‘LT₁₉₂:STa₁₃’ fusion proteins. BL21 and 1836-2 strains
were transformed with ‘LT₁₉₂:STa₁₃’ fusion plasmids
for expression of fusion 1-5 and fusion 1b-5b proteins, respectively.

| Strains | Relevant properties | Plasmid |
|---|---|---|
| 1836-2 | a porcine ETEC isolate | K88ac, estA gene |
| 8017 | negative control, pBR322/1836-2 | pBR322 |
| 8543 | LT₁₉₂ mutant strain (in 1836-2) | LT192 in pBR322 |
| 8460 | LT recombinant strain (in 1836-2) | LT in pBR322 |
| 8405 | ST₁₃ mutant strain (in 1836-2) | STa₁₃ in pBR322 |
| 8325 | STa recombinant strain (in 1836-2) | STa in pBR322 |
| 8653 | fusion 1 strain, 1836-2/pfusion-1 | LT₁₉₂-Gly:Pro-STa₁₃/pBR322 |
| 8654 | fusion 2 strain, 1836-2/pfusion-2 | LT₁₉₂-L-STa₁₃/pBR322 |
| 8670 | fusion 3 strain, 1836-2/pfusion-3 | STa₁₃-Gly:Pro-LT₁₉₂/pBR322 |
| 8672 | fusion 4 strain, 1836-2/pfusion-4 | LT₁₉₂A1-STa₁₃-LTA2-B/pBR322 |
| 8687 | fusion 5 strain, 1836-2/pfusion-5 | LT_{192A}-STa₁₃-LTB/pBR322 |
| BL21 | E. coli B F−, ompT, hsdS (rB−, mB−), gal, dcm. | |
| 8750 | fusion 1b strain, BL21/pfusion-1b | LT₁₉₂-Gly:Pro-STa₁₃/pET28α |
| 8751 | fusion 2b strain, BL21/pfusion-2b | LT₁₉₂-L-STa₁₃/pET28α |
| 8752 | fusion 3b strain, BL21/pfusion-3b | STa₁₃-Gly:Pro-LT₁₉₂/pET28α |
| 8753 | fusion 4b strain, BL21/pfusion-4b | LT_{192A1}-STa₁₃-LTA2-B/pET28α |
| 8754 | fusion 5b strain, BL21/pfusion-5b | LT_{192A}-STa₁₃-LTB/pET28α |

### II. Gene Cloning and Mutation

A. Porcine estA

The porcine STa gene (estA) was amplified by the polymerase chain reaction (PCR) using genomic DNA from a porcine ETEC field isolate 04-21018 (F18+STa+STb+Stx2e+) and designed primers STaSfcI-F and STaEagI-R (for cloning in pACYC184 vector) or STaHindIII-F and STaBamH1-R (for cloning in pUC19 vector). Each forward primer contains a SfcI or a HindIII restriction site whereas the reverse primer has an EagI or a BamH1 restriction site. PCR was performed on a PTC-100 Thermal Cycler in 50 μl of reaction containing 1×pfu DNA polymerase buffer (with Mg++), 200 nM dNTP, 0.5 μM of each forward and reverse primers, and one unit of pfu DNA polymerase. Amplified products were separated by 1.5% agarose gel electrophoresis and purified. Purified PCR products, plasmid pACYC184 and pUC19 were digested sequentially with SfcI and EagI, or HindIII and BamH1 restriction enzymes, respectively. Digested estA gene products and vectors were purified and then ligated with T4 DNA ligase. Two microliters of ligation products were introduced into 987P fimbrial E. coli construct 8227 (G58-2/pDMS167; to host plasmids with STa cloned in pACYC184) or 8795 (G58-2/pDMS158; to host plasmids with STa cloned in pUC19) competent cells by electroporation. Positive colonies were screened by PCR initially and then sequenced to ensure that the cloned gene was inserted in the correct reading frame.

To substitute the 11th, 12th, and the 13th amino acids of the pSTa toxin, specific PCR primers were designed to mutate nucleotides encoding these three residues. For an STa₁₁ toxoid gene, recombinant STa strain DNA was used as templates and the 5′ end of the estA gene was amplified by PCR using the 184EcoRV-F and the pSTa₁₁ₖ-R primers, and the 3′ end of the estA gene in another PCR with the pSTa₁₁ₖ-F (complementary to the pSTa₁₁ₖ-R primer) and pBREagI-R primers. The 5′-end and the 3′-end fragments were overlapped in a splice overlap extension (SOE) PCR to introduce a mutation at nucleotides encoding the 11th amino acid residue of the estA gene. Similarly, the estA gene at nucleotides encoding the 12th and 13th amino acids was also mutated with respective primers.

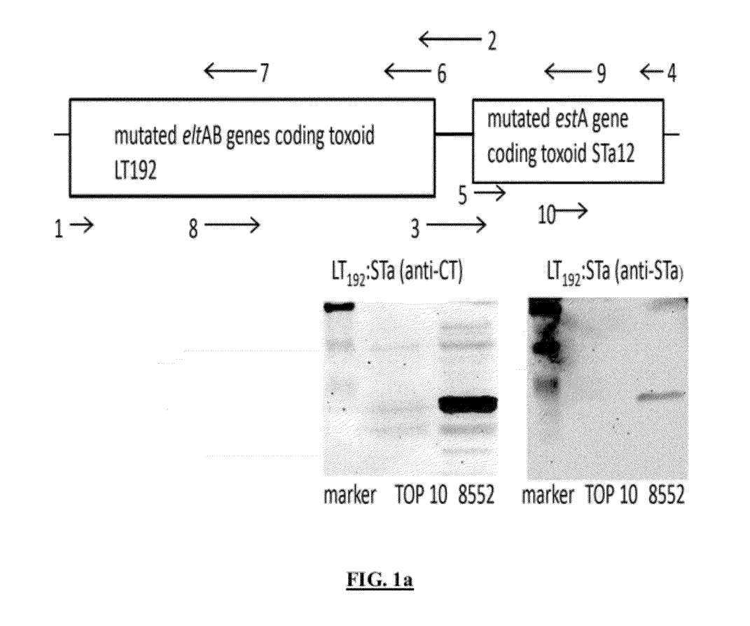

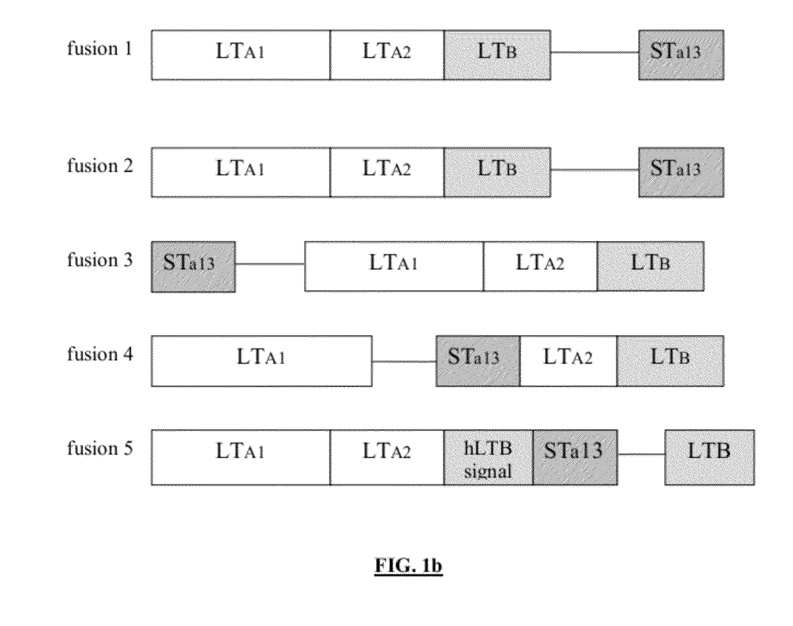

FIG. 1a demonstrates construction of porcine strain STa/LT fusion proteins. PCR primers 184EcoRV-F and LT-R amplified the entire porcine eltAB genes (without stop codon), and primers STa-F and pBREagI-R amplified the full-length porcine estA gene (without signal peptide). Primers LT₁₉₂-R and LT₁₉₂-F; complementary to LT₁₉₂-R mutated eltAB genes for LT₁₉₂, and primers mSTa12-R and mSTa12-F; complementary to mSTa12-R mutated the STa gene for an STa mutant. Primers pLT:STa-R and pLT:STa-F added a ‘Gly-Pro’ linker and genetically fused the mutated LT genes and the mutated STa gene. FIG. 1a is not proportional in gene sizes. The inserted picture at the right lower corner shows Western blot detection of a toxoid fusion using anti-CT and anti-STa antibodies. Total protein samples from TOPO cells were used as a negative control.

TABLE 2a
PCR primers used to construct porcine STa recombinant, STa mutant,
and ‘pLT₁₉₂:pSTa₁₂’ and ‘pLT₁₉₂:pSTa₁₂’ chimeric strains.

| Primers | Sequence (5'-'3) | |
|---|---|---|
| 184EcoRV-F | gtcaggcaccgtgtatgaaat (SEQ ID NO: 10) | plasmid pACYC184 sequence at the EcoRV site |
| pBREagI-R | gtccctgatggtcgtcatct (SEQ ID NO: 11) | plasmid pACYC 184 sequence downstream |
| | | at EagI site |
| STaSfcI-F | gtgaaacaacctgtaggga (SEQ ID NO: 12) | amplify native estA gene, cloned |
| | | into pACYC184 |
| STaEagI-R | gtggagccggccgaaaca (SEQ ID NO: 13) | amplify native estA gene, cloned |
| | | into pACYC184 |
| STaHindIII-F | tgcaaaataagcttaactaatctc (SEQ ID NO: 14) | amplify native estA gene, cloned into pUC19 |
| STaBamH1-R | gtggagccggatccaacag (SEQ ID NO: 15) | amplify native estA gene, cloned into pUC19 |
| pSTa_{11K}-F | 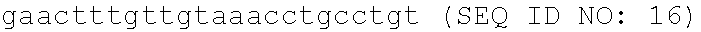 | pairs with pBREagI-R to mutate the |
| | | 11^{th} amino acid |
| pSTa_{11K}-R | 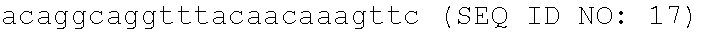 | pairs with 184EcoRV-F to mutate the |
| | | 11^{th} amino acid |
| pSTa_{12F}-F | 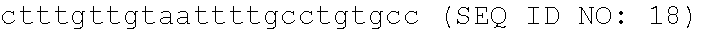 | pairs with pBREagI-R to mutate the |
| | | 12^{th} amino acid |
| pSTa_{12F}-R | 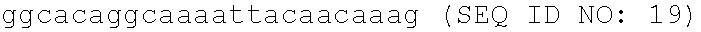 | pairs with 184EcoRV-F to mutate the |
| | | 12^{th} amino acid |
| pSTa_{13Q}-F | 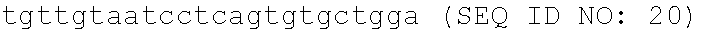 | pairs with pBREagI-R to mutate the |
| | | 13^{th} amino acid |
| pSTa_{13Q}-R | 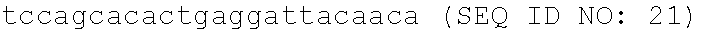 | pairs with 184EcoRV-F to mutate the |
| | | 13^{th} amino acid |
| pLT:STa-F | gcaatcagt gggccggggccc atgaacaacttttactgctg | 3′ end of LT + linker + 5′ end of STa |
| | (SEQ ID NO: 22) | |
| pLT:STa-R | gttgttcat gggccccggccc actgattgccgcaattgaattgg | 5′ end of STa + linker + 3′ end of LT |
| | (SEQ ID NO: 23) | |
| STa₁₂NS-R | 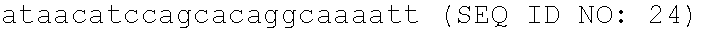 | STa₁₂ mutant without stop codon, for |
| | | TA cloning |
| ST₁₃NS-R | 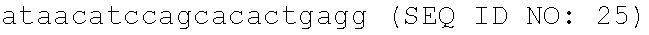 | STa₁₃ mutant without stop codon, for |
| | | TA cloning |
| LT-R | gtttttcatactgattgccccaattg (SEQ ID NO: 26) | to amplify LT_{AB} genes without stop codon |
| STa-F | aacaacacattttactgctgtg (SEQ ID NO: 27) | to amplify the STa gene without signal |
| | | peptide |
| hLT-F | atgattgacatcatgttgcatatagg (SEQ ID NO: 28) | to amplify ‘LT₁₉₂:mSTa’ fusion genes, |
| | | for TA cloning |

Nucleotides in shadow indicated the target amino acid to be mutated for STa toxoids, nucleotides underlined are the enzyme restriction site, and nucleotides in italic are the “Gly-Pro-Gly-Pro” (SEQ ID NO: 61) linker. Primers pLT:STa-F and pLT:STa-R were used to fuse the mutated porcine eltAB (coding pLT₁₉₂ protein) with mutated porcine estA (coding STa₁₂ or STa₁₃ protein) genes.

B. Human estA and LTAB

The STa gene (estA) and LTAB genes (eltAB) were PCR amplified from H10407 genomic DNA with designed primers STaNheI-F and STaEagI-R, and LTNhe-F and LTEagI-R, respectively. PCR was performed at a PTC-100 thermal cycler (BIORAD, Hercules, Calif.) in 50 μl of reaction containing 1×pfu DNA polymerase buffer (with Mg++), 200 nM dNTP, 0.5 μM of each forward and reverse primers, and one unit of pfu DNA polymerase (Strategene, La Jolla, Calif.). Amplified products were separated by 1.0% agarose (FMC Bioproducts, Rockland, Mass.) gel electrophoresis and purified using a QIAquick Gel Extraction Kit (QIAGEN, Valencia, Calif.). Purified PCR products (inserts) and vector pBR322 were digested with NheI and EagI restriction enzymes (New England Biolab, Ipswich, Mass.). Digested insert and vector products were ligated with T4 DNA ligase (New England BioLab). Two ml ligation products were introduced into 1836-2 competent cells by standard electroporation. Ampicillin selected colonies were initially screened by PCR and then sequenced to ensure that cloned genes were inserted in the correct reading frame.

FIG. 1b demonstrates construction of human strain Sta/LT fusion proteins. Cloned LTAB genes were mutated at the nucleotides coding the 192th amino acid residue and the cloned STa gene was mutated at the 13th amino acid in a three-step PCR. Briefly, two PCRs were carried out using pBRNheI-F with LT₁₉₂-R and LT₁₉₂-F with pBREagI-R to amplify the 5′ and 3′ of the eltAB genes, respectively. Then the two amplified products were fused using an SOE PCR to introduce a substitution at the nucleotides coding the 192^{th} amino acid for LT₁₉₂. Similarly, the STa gene was mutated with PCR using pBRNheI-F with STa13-R and STa13-F with pBREagI-R for amplification of the 5′ and 3′ end of the gene, respectively; and two amplified fragments were fused using an SOE PCR to introduce a mutation at the nucleotides coding the 13th amino acid for STa. The SOE PCR products were re-amplified and digested with NheI and EagI enzymes, and the mutated genes cloned into vector pBR322. FIG. 1b is not proportional in gene sizes.

TABLE 2b
PCR primers used to clone and mutate the eltAB (coding LT) and estA
(coding STa), and construct LT₁₉₂-STa₁₃ fusion genes. Nucleotides
underlined are the restriction site, the ‘Gly-Pro’ or the ‘L-linker’
are in italics.

| Primers | Sequence (5′-3′) | |
|---|---|---|
| STaNheI-F | cgacgtgtttgctagctaa | to amplify native & mutated estA gene, cloned |
| | (SEQ ID NO: 29) | into pBR322 (NheI/EagI) |
| STaEagI-R | gtggagccggccgaaaca | |
| | (SEQ ID NO: 13) | |
| LTNheI-F | atcctcgctagcatgttttat | to amplify native & mutated eltAB genes; pairs |
| | (SEQ ID NO: 30) | with pBREagI-R to amplify fusion1, 2, 4 & 5, |
| | | and clone into pBR322 (NheI/EagI) |
| LTEagI-R | gcgtcggccgctactagttttccatactgattgc | to amplify native & mutated eltAB genes, and |
| | (SEQ ID NO: 31) | clone into pBR322(NheI/EagI) |
| pBREagI-R | gtccctgatggtcgtcatct | |
| | (SEQ ID NO: 11) | |
| pBRNheI-F | tgctaacgcagtcaggcaccgtgtatg | to amplify cloned eltAB or estA toxoid gene for |
| | (SEQ ID NO: 32) | SOE; pairs with LTEagI-R to amplify fusion 3 & |
| | | clone into pBR322(NheI/EagI) |
| LT₁₉₂-F | aattcatcaggaacaattacagg | to mutate eltAB genes for LT₁₉₂ toxoid |
| | (SEQ ID NO: 33) | |
| LT₁₉₂-R | cctgtaattgttcctgatgaatt | to mutate eltAB genes for LT₁₉₂ toxoid |
| | (SEQ ID NO: 34) | |
| STa₁₃-F | ttgtgttgtaattttgcttgtacc | to mutate estA gene for STa₁₃ toxoid |
| | (SEQ ID NO: 35) | |
| STa₁₃-R | ggtacaagcaaaattacaacacaa | to mutate estA gene for STa₁₃ toxoid |
| | (SEQ ID NO: 36) | |
| STa₁₃:LT₁₉₂-F | gcaatcagtgggccggggcccatgaatagtagcaattac | 3′ end of LT + Gly-Pro linker + 5′ end of STa, to |
| | tgc | construct fusion 1 gene |
| | (SEQ ID NO: 37) | |
| LT₁₉₂:STa₁₃-R | actattcatgggccccggcccactgattgccgcaattga | 5′ end of STa + Gly-Pro linker + 3′ end of LT, to |
| | attgg | construct fusion 1 gene |
| | (SEQ ID NO: 38) | |
| STa₁₃-L-LT₁₉₂-F | agtgatccccgggtaccgagctcgatgaatagtagcaat | 3′ end of LT + L-linker + 5′ end of STa, to |
| | tactgc | construct fusion 2 gene |
| | (SEQ ID NO: 39) | |
| STa₁₃-L-LT₁₉₂-R | catcgagctcggtacccggggatcactgattgccgcaat | 5′ end of STa + L-linker + 3′ end of LT, to |
| | tgaattgg | construct fusion 2 gene |
| | (SEQ ID NO: 40) | |
| STa₁₃:LT₁₉₂-F | accgggtgctatgggccggggcccaatggcgacaaatt | 3′ end of STa + Gly-Pro linker + 5′ end of LT, to |
| | ataccgt | construct fusion 3 gene |
| | (SEQ ID NO: 41) | |
| LT₁₉₂:STa₁₃-R | gtcgccattgggccccggcccatagcacccggtacaag | 5′ end of LT + Gly-Pro linker + 3′ end of STa, to |
| | caaaatt | construct fusion 3 gene |
| | (SEQ ID NO: 42) | |
| LT₁₉₂A1:STa₁₃-F | tcatcaggagggccggtcgacatgaatagtagcaattact | 3′ end of LTA1 + linker + 5′ end of STa, to |
| | gctgtg | construct fusion 4 gene |
| | (SEQ ID NO: 43) | |
| STa₁₃:LT₁₉₂A1-R | gctactattcatgtcgaccggccctcctgatgaatttcca | 5′ end of STa + linker + 3′ end of LTA1, to |
| | caaccttg | construct fusion 4 gene |
| | (SEQ ID NO: 44) | |
| STa₁₃:LT₁₉₂A2-F | gggtgctatacaattacaggtgatacttgtaatg | 3′ end of STa + 5′ end of LTA2, to construct |
| | (SEQ ID NO: 45) | fusion 4 gene |
| LT₁₉₂A2:STa₁₃-R | cacctgtaattgtatagcacccggtacaagcaaaattac | 5′ end of LTA2 + 3′ end of STa, to construct |
| | (SEQ ID NO: 46) | fusion 4 gene |
| LT₁₉₂BS:STa₁₃-F | gcatacggaatgaatagtagcaattactgc | 3′ end of LTB signal peptide + 5′ end of STa, to |
| | (SEQ ID NO: 47) | construct fusion 5 gene |
| STa₁₃:LT₁₉₂B-R | actattcattccgtatgcacatagagagg | 5′ end of STa + 3′ end of LTB signal peptide, to |
| | (SEQ ID NO: 48) | construct fusion 5 gene |
| STa₁₃:LT₁₉₂B-F | gggtgctatgggccggggcccgctccccagtctattaca | 3′ end of STa + Gly-Pro linker + 5′ end of mature |
| | gaactatg | peptide of LTB, to construct fusion 5 gene |
| | (SEQ ID NO: 49) | |
| LT₁₉₂B:STa₁₃-R | ctggggagcgggccccggcccatagcacccggtacaa | 5′ end of mature peptide of LTB + Gly-Pro linker + |
| | gcaaaattac | 3′ end of STa, to construct fusion 5 gene |
| | (SEQ ID NO: 50) | |
| LT₁₉₂NheI-F3 | gtttgctagcaatggcgacaaattatac | To amplify the eltAB genes and to remove its |
| | ((SEQ ID NO: 51) | trans-membrane signal peptide, and clone into |
| | | pET28a(+) |
| STa₁₃EagI-R1 | gcgacggccgttattaatagcacccggtacaagc | To amplify and clone ‘LT₁₉₂-STa₁₃’ fusions |
| | (SEQ ID NO: 52) | (fusion 1b and 2b genes) |
| STa₁₃NheI-F2 | gtagctagcatgaatagtagcaattac | To amplify the estA gene and to remove its trans- |
| | (SEQ ID NO: 53) | membrane signal peptide, and clone into |
| | | pET28a(+) |
| LT₁₉₂BamHI-R1 | gcgtggatccctactagttttccatact | Pairs with ‘STa₁₃NheI-F2’ to amplify fusion 3b, |
| | (SEQ ID NO: 54) | pairs with ‘LT₁₉₂NheI-F3’ to amplify fusion 4b |
| | | and 5b, cloned into pET28a(+) |
| LT₁₉₂A-LL- | gaattagatccccgggtaccgagctcggctccccagtct | 3′ end of LT_{A} + L-linker + 5′ end of mature |
| LT₁₉₂B-F | attacag | peptide of LT_{B} for fusion lb, 3b and 4b genes |
| | (SEQ ID NO: 55) | |
| LT₁₉₂A-LL- | ctggggagccgagctcggtacccggggatctaattcatt | 5′ end of mature peptide of LT_{B} + L-linker + 3′ |
| LT₁₉₂B-R | ccgaattctg | end of LT_{A} for fusion 1b, 3b and 4b genes |
| | (SEQ ID NO: 56) | |
| LT₁₉₂A-SL- | gaattagggccggggcccgctccccagtctattacag | 3′ end of LT_{A} + Gly-Pro linker + 5′ end of |
| LT₁₉₂B-F | (SEQ ID NO: 57) | mature peptide of LT_{B} for fusion 2b gene |
| LT₁₉₂A-SL- | ctggggagcgggccccggccctaattcattccgaattc | 5′ end of mature peptide of LT_{B} + Gly-Pro linker + |
| LT₁₉₂B-R | tg | 3′ end of LT_{A} for fusion 2b gene |
| | (SEQ ID NO: 58) | |
| LT₁₉₂A-LL- | gaattagatccccgggtaccgagctcgatgaatagtagc | 3′ end of LT_{A} + L-linker + 5′ end of mature |
| STa₁₃-F | aattac | peptide of STa for fusion 5b gene |
| | (SEQ ID NO: 59) | |
| LT₁₉₂A-LL- | actattcatcgagctcggtacccggggatctaattcattc | 5′ end of mature peptide of STa + L-linker + 3′ |
| STa₁₃-R | cgaattctg | end of LTA for fusion 5b gene |
| | (SEQ ID NO: 60) | |

### III. Detection in Expression and Toxicity of Fusion Antigens

A. Porcine

i. STa Competitive ELISA

Overnight culture growth of an STa recombinant, each of three mutant strains, and a negative control strain were used in an STa competitive ELISA. Briefly, each strain was cultured in LB medium overnight, and culture growth was measured with optical density (OD). An equivalent amount of cells from each strain were used for subculture in 4 AA medium, and the 4 AA culture supernatants were used for ELISA. An STa ELISA plate was coated with STa ovalbumin-conjugate (1.25 ng per well) overnight at 37° C., and blocked with 2.5% casein blocking buffer (2.5% casein in 0.3 N NaOH, pH 7.0). Seventy-five microliters of culture supernatant from each strain (in triplicates) and 75 μl of anti-STa serum (1:10,000) were mixed and added to each well, followed by an incubation at 37° C. for 2 hours on a shaker (180 rpm). After three washes, plates were blotted to dry, incubated with horseradish peroxidase (HRP)-conjugated goat anti-rabbit immunoglobulin (IgG) (1:10,000) at 37° C. for 1 hr, and reaction of bound IgG with ABTS [2,2′-azinobis93-ethylbenzthiasoline sulfonic acid)] substrate was measured at 405 nm.

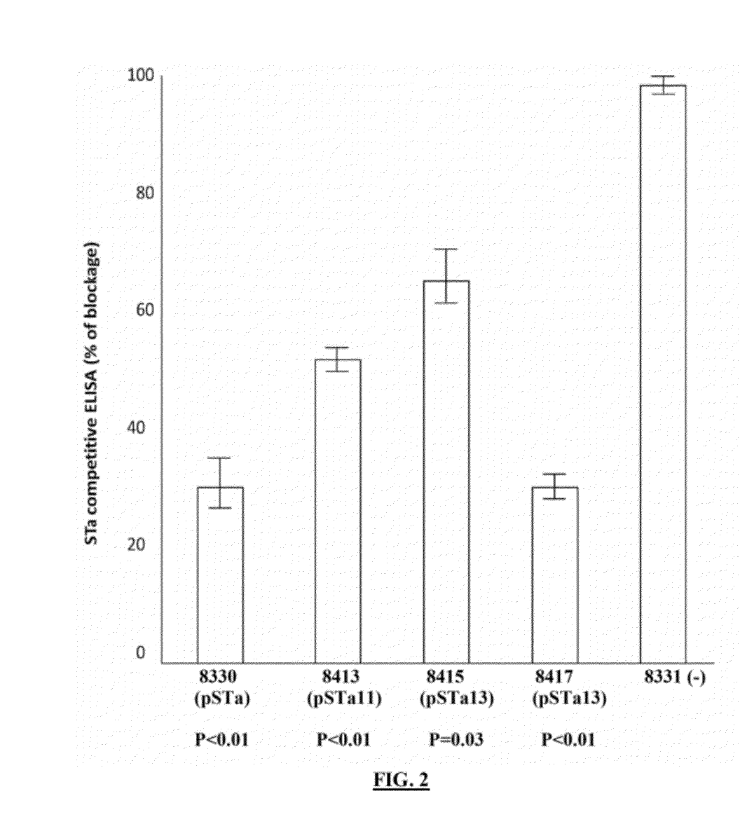

As demonstrated by FIG. 2, STa proteins were expressed by all STa recombinant and mutant strains. FIG. 2 shows the results of the ELISA among the STa recombinant 8330 (STa) and mutant strains 8413 (STa₁₁), 8415(STa₁₂) and 8417(STa₁₃). 1.25 ng STa-ovalbumin conjugate was coated on each well, and anti-STa serum (1:10,000) was used as the primary antibodies and horseradish peroxidase-conjugated goat anti-rabbit immunoglobulin (IgG; 1:10,000) was used as the secondary antibodies. Optical densities were measured at 405 nm. STa proteins were detected in the STa recombinant and each of the three mutant strains. Overnight-grown 4AA culture supernatant samples from the recombinant STa (8330), the mutant STa₁₁ (8413), STa₁₂ (8415), STa₁₃ (8417), and the negative control (8331) strains were used to compete with 1.25 ng of synthetic STa-ovalbumin conjugates (in 75 μl ELISA dilution buffer, pre-coated in each well) for anti-STa antibodies in an STa competitive ELISA. ELISA results indicated that pre-coated synthetic STa-ovalbumin conjugates bound 30%, 50%, 63%, and 29.2% of the anti-STa serum after competing with STa proteins from 8330, 8413, 8415, and 8417 that differed significantly from the negative control 8331 (97%). By referring to the curve generated with a series of STa synthetic peptide standards (0, 0.01, 0.05, 0.10, 0.50, 1.0, 5.0, and 10 ng in 75 μl STa ELISA buffer), STa toxoid proteins expressed in 8413, 8415 and 8417 strains were estimated at a range from 1.3 ng/ml to 13 ng/ml.

ii. Cyclic GMP ELISA to Detect Toxicity of STa Proteins

Toxicity of the recombinant and mutated STa proteins was tested in stimulation of intracellular cGMP levels in T-84 cells (ATCC #CCL-248). Bacterial culture growth supernatant was used for cGMP ELISA using a direct cyclic GMP enzyme immunoassay kit (acetylated version). Briefly, 1×10⁵ T-84 cells were seeded and cultured in each well of a 24-well plate. After removing the Dulbecco's modified Eagle medium (DMEM/F12), 75 μl overnight culture growth (in 4AA medium) supernatant from each strain (in duplicate) was added to each well. Cells were lysed with 200 μl (per well) 0.1 M HCl after a 2 hour incubation. One hundred microliters of cell lysate was mixed with the conjugates and antibody reagents. The mixture was added to each well of a supplied EIA plate. After incubation on a shaker (500 rpm) at room temperature for 2 hours, plates were washed, dried, and reacted with pNpp (p-Nitrophenyl Phosphate, disodium salt) substrate solution. The OD was measured at 405 nm after 20 minutes of development.

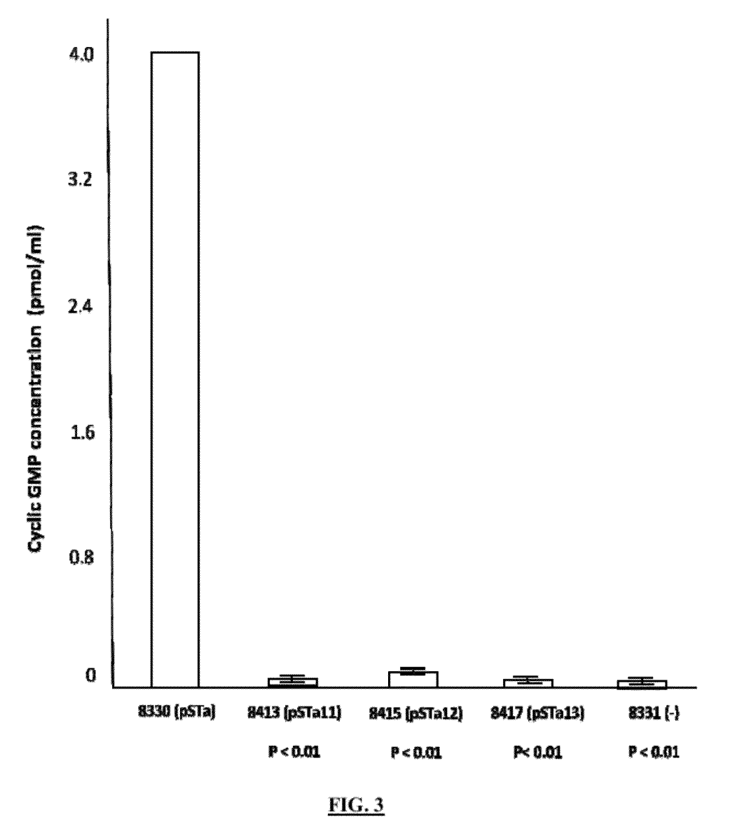

STa toxoid proteins expressed in all 3 mutant strains showed significant reduction in toxicity from the control recombinant strain. Results from cGMP ELISA (acetylated version) indicated that intracellular cGMP concentrations in T-84 cells stimulated by 8330, 8413, 8415, 8417, and 8331 culture were 4±0, 0.0185±0.0065, 0.043±0.015, 0.017±0.0015, and 0.012±0.0005 pmole/ml, respectively (FIG. 3). All mutant strains showed low or no stimulation on cGMP levels compared to the recombinant strain, suggesting these STa toxoid proteins had toxicity significantly reduced. Statistical analysis indicated that stimulation of intracellular cGMP in T84 cells by three STa toxoids was significantly lower compared to that from the recombinant STa (p<0.01, p<0.01, p<0.01). Among 3 mutant strains, mutant STal2 (8415) showed the highest stimulation of cGMP level (43 fmole/ml), whereas STa₁₃ (8417) had the lowest stimulation of cGMP (17 fmole/ml), which was not significantly different from the negative control strain 8331 (12 fmole/ml; p=0.25).

iii. Porcine Ligated Intestinal Loops

Biological activity of the recombinant and mutant STa porcine antigenic polypeptide was examined in a porcine ligated loop assay. Fifteen loops were made through ileum and jejunum sections, and 2×10⁹ CFUs of culture growth from the recombinant, each mutant strain, and a control strain were injected into each ligated loop. After 8 hours post-inoculation, the length of each loop (cm) and amount of fluid accumulated in each loop (gram) were measured. The ratio of fluid accumulation to the loop length (g/cm) was calculated as an index of enterotoxic activity.

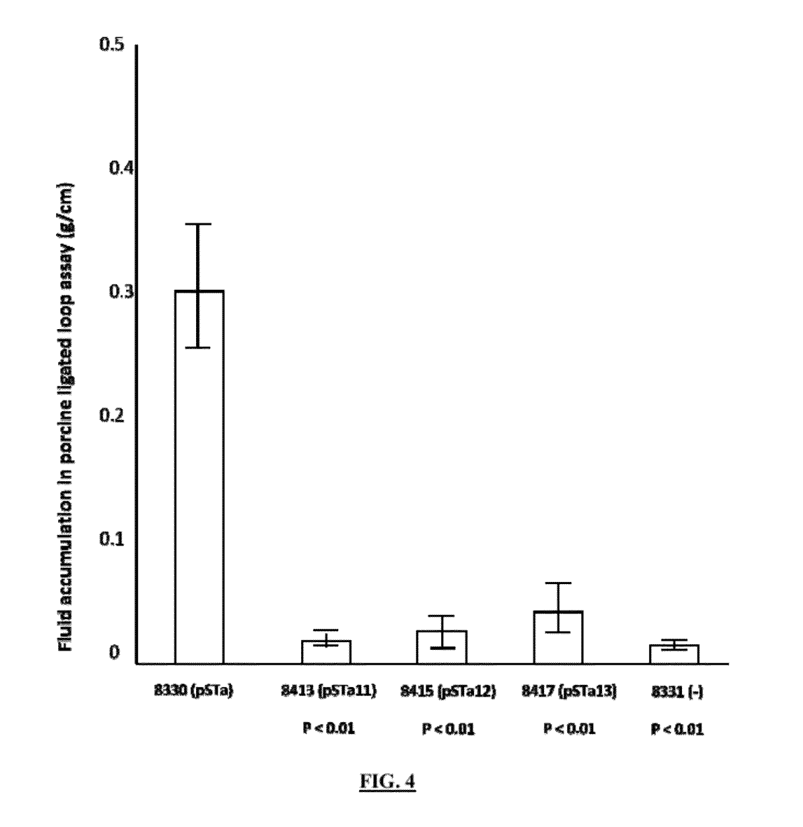

Results from porcine ligated gut loop assay indicated that the STa toxoid proteins expressed by all 3 mutant strains did not stimulate fluid secretion. After an 8 hour incubation, only loops incubated with the recombinant STa strain (8330) showed fluid accumulation (0.3 g/cm), whereas loops incubated with 8413, 8415 and 8417 mutant strains had 0.02, 0.03, 0.04 g/cm fluid accumulated, respectively. Fluid accumulation in loops incubated with the mutant strains and the negative control was significantly different from that in loops inoculated with the recombinant strain (p<0.01) (FIG. 4).

FIG. 4 2×10⁹ CFUs of 8330 (STa), 8413 (STa₁₁), 8415(STa₁₂), 8417(STa₁₃) or a negative control 8331(−) strain were incubated in each loop (three repeats). After an 8 hour incubation, fluid accumulated in each loop was measured, and a ratio of the accumulated fluid (gram) and the loop length (cm) was used as an index.

iv. Animal Challenge Studies

Twenty 3-day old gnotobiotic piglets were randomly divided into five groups. Each group was orally inoculated with 3×10⁹ CFUs overnight-grown culture of each of the three mutant strains, the STa recombinant, or the negative control strain. During the 24-hour post-inoculation period, piglets were closely monitored for clinical signs of disease, including vomiting, diarrhea, dehydration, lateral recumbency, and lethargy.

To test whether expressed STa toxoids were safe to young pigs, three-day old, 987P receptor positive gnotobiotic pigs were challenged with the recombinant or each STa mutant strain. During 24 hours post-inoculation, only pigs in the group challenged with the STa recombinant strain developed diarrhea, whereas pigs inoculated with STa mutant strains remained completely healthy. To affirm all piglets possessing 987P receptors, we collected small intestinal samples from each challenged pig at necropsy to prepare brush border vesicles for adherence assay. Brush border bacterial adherence assay indicated that all challenged pigs expressed 987P receptors. In addition, quantitative culture studies showed that colonization of mutant STa constructs ranged from 8.0×10⁸ to 1.7×10⁹ CFUs per gram of ileum tissue, suggesting all mutant strains were well colonized in small intestines of the challenged pigs.

B. Human

i. ELISA

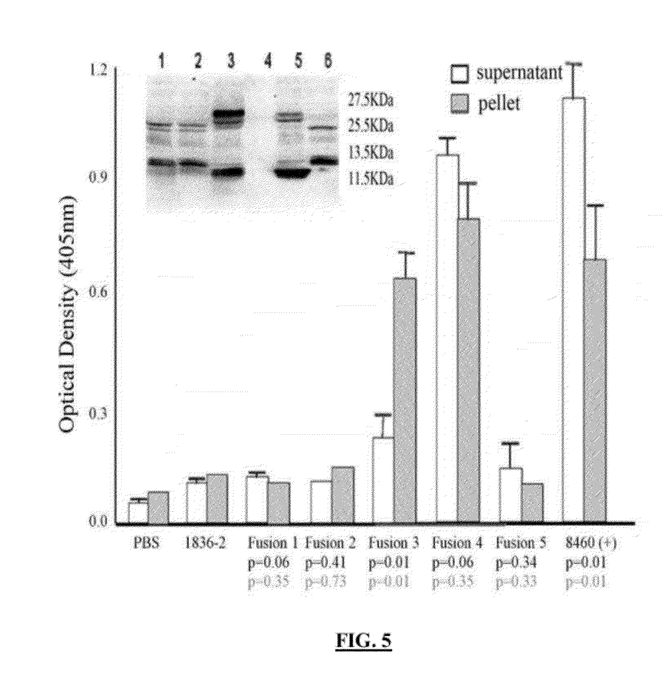

Expressed STa₁₃ and LT₁₉₂ human strain proteins could be detected using an STa competitive ELISA and a GM1 ELISA (FIG. 5). Toxicity in STa₁₃ and LT₁₉₂ was tested in T-84 cells for stimulation of intracellular cyclic GMP and AMP by using cGMP and cAMP EIA kits (Correlated EIA, Assay Design, MI), respectively. LT proteins were detected in the LT₁₉₂ mutant strain 8543 with the GM1 ELISA and STa proteins in the STa₁₃ mutant strain 8405 using an STa competitive ELISA. Total protein extracts from overnight culture growth of 8543 were used in GM1 ELISA, and supernatant of overnight growth of 8405 strain was used in STa competitive ELISA. GM1 ELISA data indicated that LT₁₉₂ protein was expressed in the mutant strain at a level (OD=1.026±0.11) similar to the native LT protein from the LT recombinant strain 8460 (OD=1.082±0.05; p<0.01). STa competitive ELISA showed that the STa₁₃ protein was expressed, as 53% to 56% of anti-STa antibodies were blocked from binding to coated STa-ovalbumin conjugates by the STa₁₃ proteins from the 8405 strain.

ii. Porcine Ligated Intestinal Loops

In addition, toxic activity of LT₁₉₂ and STa₁₃ was examined in porcine ligated gut loop assay. Briefly, 20 loops from the ileum and jejunum sections of a 5-day old piglet were prepared, and 2×10⁹ CFUs of each mutant strain were injected into each loop (4 replicates). After 8 h postinoculation, the amount of fluid accumulated in each loop (g) and the length of the loop (cm) were measured. The g/cm ratio was calculated as the index of enterotoxic activity.

LT1₉₂ and STa₁₃ proteins no longer stimulated fluid secretion. LT₁₉₂ and STa₁₃ mutant strains did not stimulate fluid accumulation in ligated porcine gut loops or an increase of intracellular cAMP and cGMP levels in T-84 cells. After 8 h postinoculation, fluid accumulated in loops inoculated with overnight culture growth of 8325 (STa), 8460 (LT), 8405(STa13), 8543(LT192), and a negative control 8017 (55) were 0.242±0.145, 0.198±0.16, 0.05±0.04, 0.05±0.02, and 0.025±0.003 (g/cm), respectively. When overnight growth supernatant of 8405 or 8543 was incubated with T-84 cells, no increase in levels of either intracellular cAMP or the cGMP was detected in T-84 cells.

IV. Construction of pLT:pSTa Chimeric Genes

A. Porcine ‘pLT₁₉₂:pSTa₁₂’ and ‘pLT₁₉₂:pSTa₁₃’

Recombinant porcine eltAB genes that encode pLT toxin and mutated eltAB genes encoding pLT₁₉₂ strains have been cloned. A ‘Gly-Pro-Gly-Pro’ (SEQ ID NO: 61) linker was used to connect the mutated eltAB and estA genes. PCR primers pLT:STa-R and pSTa:LT-F were specifically designed so that they contained nucleotides of the 3′ end of the eltAB genes (without the stop codon), the linker, and the 5′ end of the estA gene (without the signal peptide). A PCR using primer 184EcoRV-F and pLT:STa-R and plasmid pLT₁₉₂ as the template amplified the mutated eltAB genes, the linker, and the 5′ end of the mutated estA gene. A second PCR using pSTa:LT-F and pBREagI-R primers and DNA from the STa mutant plasmid pSTa₁₂ generated the fragment covering the 3′ end of the mutant eltAB genes (no stop codon), the linker, and the estA gene of mutant STa₁₂ (no signal peptide). A SOE PCR connected the mutated eltAB and the mutated estA genes with the linker for a chimeric gene (pLT₁₉₂:pSTa₁₂). The resultant chimeric gene was further amplified with 184EcoRV-F and pBREagI-R primers and then digested with SfcI and EagI enzymes. Digested products were purified and cloned into vector pACYC184 at the SfcI and EagI sites with T4 DNA ligase. Two microliters of T4 ligation products were introduced into 8227 (G58/987P) host cells using electroporation. Positive colonies were screened by PCR and then DNA sequenced to ensure the cloned ‘pLT₁₉₂:pSTa₁₂’ fusion gene was inserted in reading frame.

Similarly, a ‘pLT₁₉₂:pSTa₁₃’ chimeric gene was constructed using plasmid pSTa₁₃ as a template. The ‘pLT₁₉2:pSTa₁₃’ chimeric gene was also cloned into vector pACYC184 and expressed in 8227 host cells. In addition, chimeric genes ‘pLT₁₉₂:pSTa₁₂’ and ‘LT₁₉₂:pSTa₁₃’ were amplified by hLT-F paired with STa₁₂NS—R and STa₁₃NS—R, and cloned into the TA clone vector pBAD-TOPO and expressed in TOPO 10 cells for protein purification by using his-tag.

B. Human

After verification of expression and low toxicity from the expressed proteins, the LT₁₉₂ and STa₁₃ genes were genetically fused. Specific PCR primers were designed to genetically fuse the STa₁₃ gene at the 3′ end of the LT₁₉₂ genes with a ‘Gly-Pro’ linker or a longer ‘L-linker’, the 5′ end of the LT₁₉₂ genes with the ‘Gly-Pro’ linker, the 3′ end of the A1 peptide of the LT₁₉₂ genes with a ‘SalI-linker’ or at the 5′ end of the eltB gene (coding the LTB subunit) with the ‘Gly-Pro’ linker, for five LT₁₉₂-STa₁₃ fusion genes designated as fusion 1-5 genes. All fusions were constructed using three-step PCR. Resultant fusion genes were cloned into vector pBR322 and expressed in 1836-2 cells.

These five fusion genes were also cloned into vector pET28α to be expressed as 6×His-tagged proteins for protein purification. In addition, fusion genes cloned into pET28α had the nucleotides coding the trans-membrane peptides removed. To clone each fusion gene into pET28 vector and to delete nucleotides coding trans-membrane signal peptides, PCRs were performed using two specifically designed external primers, δ 5′ end PCR primers with a NheI site and the 3′ end primers with a BamHI site. Each amplified fusion gene was digested with NheI and BamHI enzymes, and cloned into vector pET28α. To remove nucleotides coding trans-membrane signal peptides, PCR using two internal primers that included 10-15 nucleotides downstream of the signal peptide and 20 nucleotides upstream of the same signal peptide was conducted. Similarly, one PCR using the 5′ end external forward primer and an internal reverse primer amplified the fragment upstream of the target trans-membrane peptide, and a second PCR using the internal forward primer and the external 3′ end reverse primer amplified the fragment downstream of the same signal peptide. The two amplified products were overlapped in an SOE PCR resulting in a fusion without nucleotides coding trans-membrane signal peptides. The overlapped products were further amplified by PCR with the two external primers, and digested with NheI and BamHI enzymes. Digested fusion gene products were cloned into pET28α vector. BL21α cells were transformed with plasmids expressing each 6×His-tagged LT₁₉₂-STa₁₃ fusion protein that also had trans-membrane signal peptide removed. Kanamycin selected colonies were screened with PCR and DNA sequencing, and resultant strains were designated as fusion 1b-5b strains.

### V. Detection of LT and STa in Fusion Proteins

A. Porcine

The ‘pLT₁₉₂:pSTa₁₂’ and ‘pLT₁₉₂:pSTa₁₃’ constructs were grown in Casamino acids and yeast extract broth with lincomycin (45 μg/ml) and ampicillin (50 μg/ml) overnight at 37° C. The overnight-grown culture was centrifuged at 3000×g for 20 min, and pellets were collected for total protein preparation using bacterial protein extraction reagent (B-PER, in phosphate buffer).

Thirty microliters of each total protein sample were used to detect LT and STa in a sodium dodecyl sulfate-polyacrylamide gel electrophoresis (SDS-PAGE) and immuno-blot assay. Transferred membrane blotting was blocked with 2% fat-free milk overnight at 4° C. and then incubated with anti-CT (1:3000) and anti-STa sera (1:5000), respectively. After three washes, the membranes were incubated with HRP-conjugated goat anti-rabbit IgG (1:5,000) for 1 hour. After a final round of washes, peroxidase bound to the fusion proteins on the membranes were detected with chemiluminescence.

B. Human

LT₁₉₂-STa₁₃ fusion proteins expressed in constructed fusion 1-5 strains and the 6×His-tagged LT₁₉₂-STa₁₃ fusion proteins by fusion 1b-5b strains were examined in SDS-PAGE and GM1-ELISA. Fusion 1-5 strains were grown overnight at 37° C. in 10 ml LB medium with ampicillin (50 μg/ml). Equivalent amounts of overnight culture growth (calculation based on cell optical density) were centrifuged at 3000×g for 20 min. Supernatant samples were collected, and pellets were saved and resuspended into 1 ml bacterial protein extraction reagent (B-PER, in phosphate buffer; Pierce, Rockford, Ill.) for total protein extraction. Thirty microliters of total protein extracts from each strain was analyzed in a standard sodium dodecyl sulfate-polyacrylamide gel electrophoresis (SDS-PAGE) and immuno-blot assay. Rabbit anti-CT (1:3300; Sigma) and anti-STa sera (1:5000; Dr. Robertson laboratory) were used to detect LT and STa, respectively. Horseradish peroxidase (HRP)-conjugated goat anti-rabbit IgG (1:5000; Sigma) antibodies were used as the secondary antibodies. Peroxidase bound to the fusion proteins on the membranes was detected with a SuperSignal West Pico chemiluminescent substrate kit (Pierce). Similarly, the 6×His-tagged fusion proteins without trans-membrane signal peptides expressed in 1b-5b strains were also examined for expression of LT and STa in SDS-PAGE by using anti-CT and anti-STa antiserum.

Data from Western blot showed that proteins of 11.5 KDa (LTB subunit), 13.5 KDa (LTB-STa₁₃, STa₁₃-LTB), 25.5 KDa (LTA subunit), and 27.5 KDa (STa₁₃-LTA, LTA-STa₁₃) were detected by anti-CT antibodies (FIG. 5, inserted image). When anti-STa antiserum was used, only the 13.5 KDa proteins (LTB-STa₁₃, STa₁₃-LTB) from fusion 1, fusion 2 and fusion 5 strains were detected as well as the 27.5 KDa proteins (STa₁₃-LTA, LTA-STa₁₃) from fusion 3 and fusion 4 strains. Extracted 6×His-tagged fusion proteins from fusion 1b-5b strains were also separated in 10% SDS-PAGE gel, and detected with rabbit anti-CT and anti-STa antisera. The 6×His-tagged fusion 1b-5b proteins, over 40 KDa (monomeric LTAB₁₉₂-STa₁₃ plus a 6×-His tag), were detected in each strain by both anti-CT and anti-STa antibodies.

Fusion proteins were also detected in GM1 ELISA using anti-CT antiserum. GM1 ELISA data showed lower GM1 binding activity from the fusion proteins that had the STa₁₃ fused at the B subunit of LT₁₉₂ was detected (FIG. 5). The OD values were 0.119±0.02 and 0.122±0.02 in the pellet and supernatant samples for fusion 1 strain, and OD values in the pellet and supernatant of fusion 2 strain were 0.142±0.02 and 0.120±0.04. These OD values (of fusion 1 and fusion 2 strains) were not significantly different compared to those in the negative control strain (0.135±0.0e in pellet, 0.100±0.01 in supernatant). Student t-test calculated that the p values were 0.35, 0.73 when the pellet samples of fusion 1 and 2 strains were compared to that of the negative control strain, and 0.06 and 0.41 when their supernatant samples were compared (FIG. 2). GM1 binding activity was also detected at a lower level when an STa₁₃ was fused after the signal peptide of the B subunit from fusion 5 strain (0.115±0.02 in pellet, 0.142±0.08 in supernatant), that was not significantly different compared to the negative control sample (p=0.34, 0.30) (FIG. 5).

VI. Purification and Immunization with Purified Fusion Proteins.

A. Porcine

Fusion proteins ‘pLT₁₉₂:pSTa₁₂’ and ‘pLT₁₉₂:pSTa₁₃’ expressed in E. coli TOPO 10 cells were purified using B-PER and Ni-NTA Agarose. Briefly, overnight culture growth (in Casamino acid and yeast medium) was centrifuged, and resultant pellets were lysed in B-PER reagent and then sonicated. Total proteins in cell lysis were incubated with Ni-TNA resin, followed by washes and elution. The 6×His-tagged TA cloned fusion proteins were purified and stored at −20° C. until use.

Two adult rabbits were immunized intramuscularly (IM) with 100 μg of purified ‘pLT₁₉₂:pSTa₁₂’, and another two rabbits with purified ‘pLT₁₉₂:pSTa₁₃’ fusion proteins, in an equal volume of Freund's incomplete adjuvant. Two booster injections were followed at biweekly intervals. One rabbit without immunization served as the negative control. Blood and fecal samples were collected before and 14 days after each immunization. Collected serum and resuspended fecal samples were stored at −80° C. until use.

B. Human

The 6×His-tagged LT₁₉₂-STa₁₃ fusion proteins expressed by fusion 1b-5b strains were purified and used to immunize mice. Expressed 6×His-tagged proteins were extracted using B-PER (Pierce), and purified to an estimated purity greater than 90% using nickel affinity chromatography. Briefly, overnight culture growth was harvested, and resultant pellets were lysed in B-PER reagent and briefly sonicated. Total protein extracts from cell lysates were incubated with Ni-TNA resins, and the 6×His-tagged fusion proteins were extracted according to the protocol of batch purification of 6-His tagged proteins from E. coli under native condition (QIAGEN, Valencia, Calif.). Purified proteins were stored at −80° C. until use.

Three female adult BALB/c mice (Harlan, Indianapolis, Ind.) per group were immunized intraperitoneally (IP) with each purified 6×His-tagged 1b, 2b, 3b, 4b, or 5b fusion protein. One hundred μg fusion proteins, in an equal volume of Freund's incomplete adjuvant (Sigma, St. Louis, Mo.), was injected to each mouse in the group. Two booster injections were followed at biweekly intervals. One group injected with saline was used as the negative control. Blood and fecal samples were collected from each mouse before immunization and 14 days after each immunization. Mice were sacrificed two weeks followed the final booster injection. Collected blood samples were left to coagulate at room temperature for 30 min, and followed by centrifugation at 8,000 rpm to collect serum. In addition, the intestines of each mouse were washed with 1 ml PBS by gently rubbing the intestines 2-3 times and collecting the washing samples. Collected serum, fecal resuspension, and intestinal washes were stored at −80° C. until use. All animal studies in this project complied with the Animal Welfare Act, followed the Guide for the Care and Use of Laboratory Animals (21a), and were approved and supervised by South Dakota State University's Institutional Animal Care and Use committee.

### VII. Antibody Titration and Neutralization

A. Porcine

Cholera toxin (CT) and STa ovalbumin-conjugates were used as antigens to titrate anti-LT and anti-STa antibodies from rabbit serum and fecal samples, respectively. For anti-LT₁₉₂ antibody titration, an ELISA plate was coated with GM1 (400 ng/well) as GM1-ELISA. Rabbit antisera (1:50 diluted in PBS; in triplicates) were used as the primary antibodies (in a binary dilution), and HRP-conjugated goat-anti-rabbit IgG as the secondary antibodies. To titrate anti-STa antibodies, an ELISA plate was coated with STa ovalbumin-conjugates (1.25 ng/well), rabbit anti-serum or anti-fecal antibody samples (1:50 diluted in STa ELISA buffer; in triplicates) were used as the primary antibodies and HRP-conjugated goat-anti-rabbit IgG or IgA as the secondary antibodies. The OD was measured at 405 nm after 20 min of development in peroxidase substrates. The titration end-point was determined as the reciprocal of the interpolated dilution giving an OD unit above 0.4 after subtraction of background. Antibody titers were expressed as the log 10 of the reciprocal dilution.

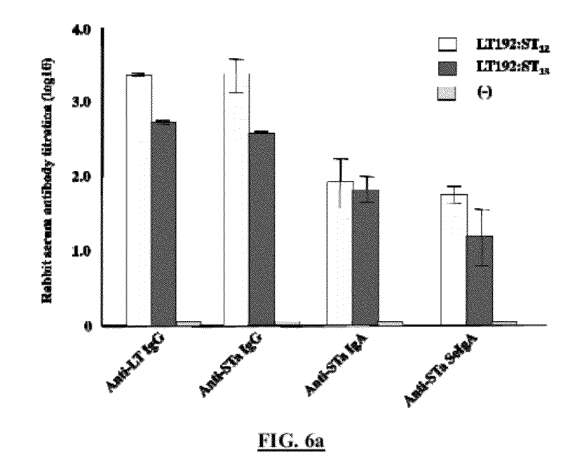

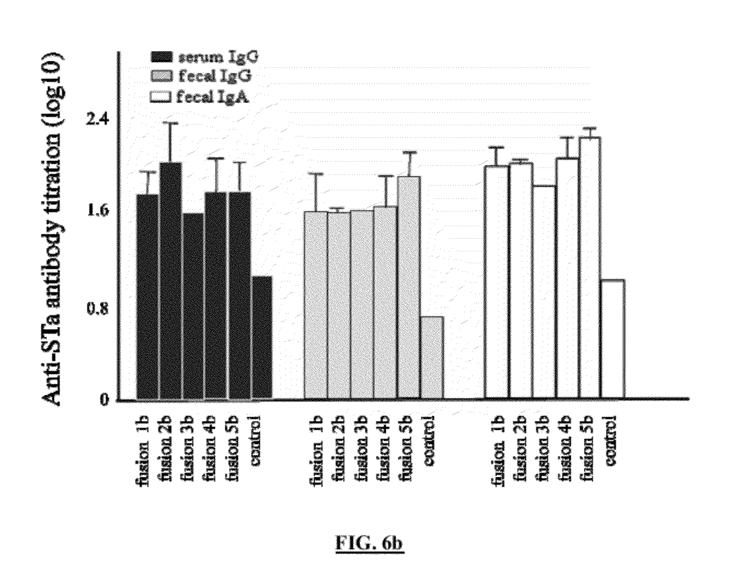

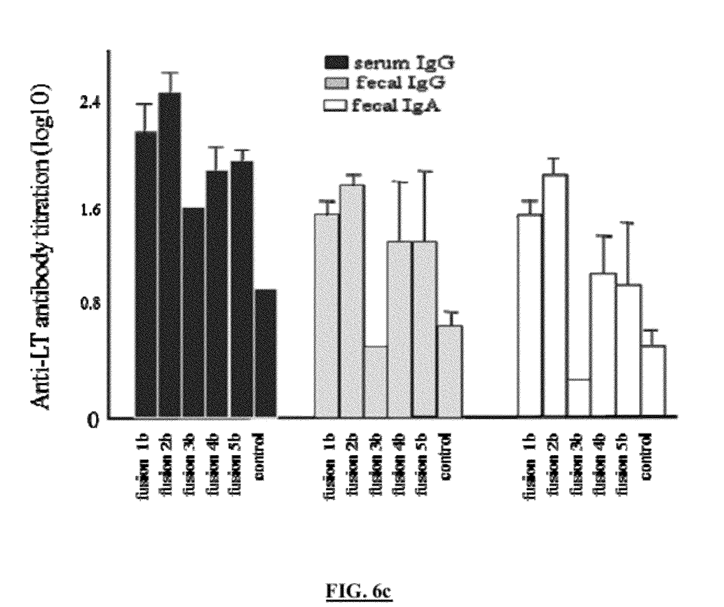

LT and STa toxoid fusions enhanced STa immunogenicity. In this study, toxoids pSTa₁₂ and pSTa₁₃ were selected to construct LT and STa toxoid fusion proteins. The pSTa₁₂ had the lowest recognition to anti-STa antiserum but stimulated the highest cGMP level in T-84 cells among the three toxoids. In contrast, pSTa₁₃ was the best in recognition of anti-STa antibody and showed a lower stimulation of intracellular cGMP level. Both LT and STa toxoids in ‘pLT₁₉₂:pSTa₁₂’ and ‘pLT₁₉₂:pSTa₁₃’ fusions were recognized by anti-CT and anti-STa antisera (inserted images in FIG. 1). Antibody titration of serum samples from the rabbits immunized with purified ‘pLT₁₉₂:pSTa₁₂’ and ‘pLT₁₉₂:pSTa₁₃’ fusion antigenic polypeptides showed 3.33±0.23 and 2.59±0.01(in log 10) titers of anti-STa IgG antibodies, and 1.92±0.34 and 1.83±0.17 titers of anti-STa IgA antibodies, respectively (in log 10; FIG. 6a). Anti-STa antibodies were detected in fecal samples as well. Anti-fecal anti-STa se-IgA antibodies in rabbits immunized with ‘pLT₁₉₂:pSTa₁₂’ and ‘pLT₁₉₂:pSTa₁₃’ fusion antigenic polypeptides were 1.75±0.14 and 1.26±0.40, respectively. As expected, anti-LT antibodies (IgG) were detected at high titers (3.33±0.02, 2.71±0.01; in log 10) (FIG. 6a).

Neutralization of CT toxin by rabbit antiserum and anti-fecal antibodies was examined using a cAMP EIA and T84 cells. For anti-STa antibody neutralization, a cGMP EIA was used as well as T84-cells. T-84 cells were cultured and 10 ng CT or 2 ng STa toxin (diluted in 150-μl DMEM/F12 medium) was incubated with 150 μl anti-sera or anti-fecal (1:50 dilution in DMEM/F12 medium, in triplicates) at room temperature. After 1 hour incubation, the mixture (150 μl CT or STa toxin dilution and 150 μl of diluted anti-sera or anti-fecal sample) was added to each well, and the plate was further incubated at 37° C. in 5% CO₂ for 2 hours. After another wash, the cells were lysed with 0.1M HCl (200 μl per well), and then neutralized with 0.1 M NaOH. The cell lysate was collected with a centrifugation at 660×g for 10 min at room temperature. Resultant supernatants were tested for intracellular cAMP or cGMP levels.

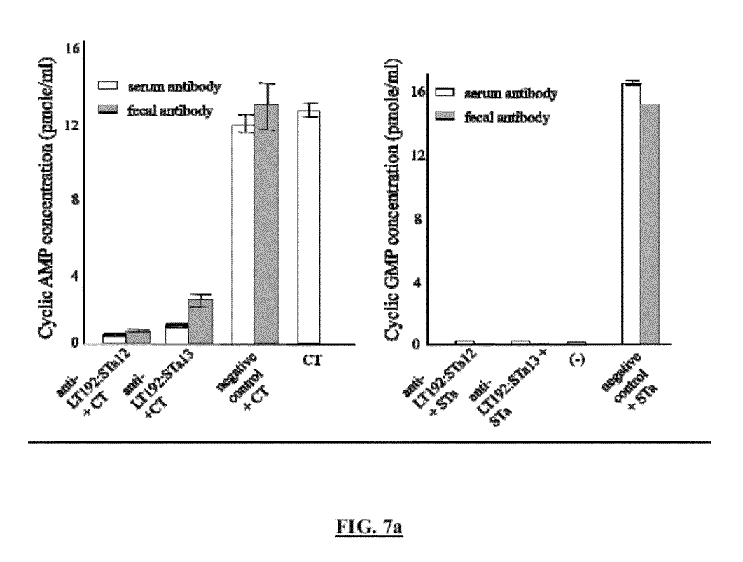

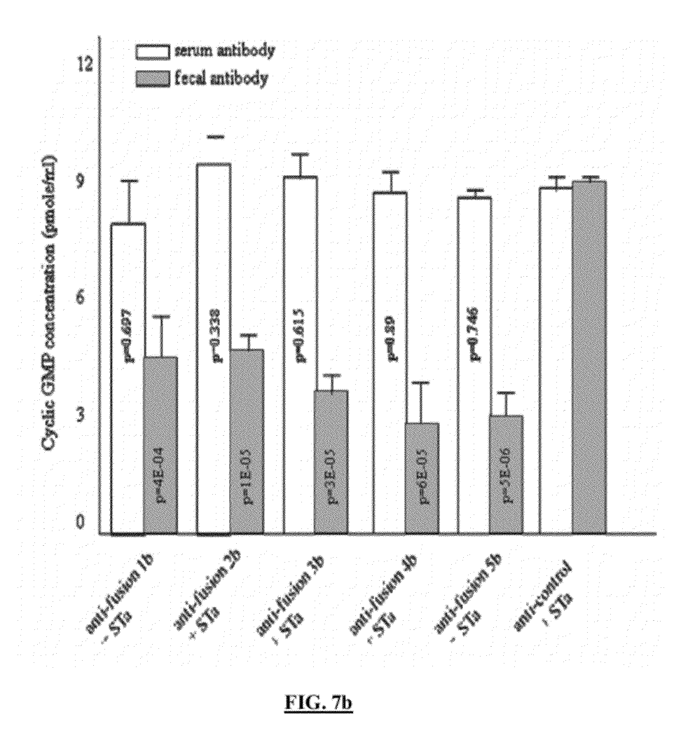

Anti-LT and anti-STa antibodies neutralized CT and STa toxins. CT toxin (10 ng) was unable to stimulate an increase of intracellular cAMP level in T-84 cell after being incubated with serum or fecal antibodies from rabbits immunized with ‘pLT₁₉₂:pSTa₁₂’ and ‘pLT₁₉₂:pSTa₁₃’ fusion antigenic polypeptides. In contrast, serum or fecal samples from the negative control rabbit did not prevent CT toxin from increasing cAMP levels in T-84 cells (FIG. 7a). Cyclic AMP concentrations in cells treated with a mixture of CT and anti-‘pLT₁₉₂:pSTa₁₂’ or anti-'pLT₁₉₂:pSTa₁₃′ serum were 0.4±0.01 and 0.52±0.04 pmole/ml, respectively; whereas the cAMP concentrations in cells treated with CT only or CT mixed with the serum sample from the negative control rabbit were 12±0.25 and 12.7±0.2 pmole/ml. Similarly, after incubation with anti-‘pLT₁₉₂:pSTa₁₂’ and anti-‘pLT₁₉₂:pSTa₁₃’ serum or fecal samples, 2 ng purified STa toxin did not increase intracellular cGMP levels in T-84 cells (FIG. 7a). The intracellular cGMP concentration in cells treated with STa mixed with anti-‘pLT₁₉₂:pSTa₁₂’ or anti-‘pLT₁₉₂:pSTa₁₃’ antiserum were 0.17±0.005 and 0.16±0.004 pmole/ml. These cGMP levels were significantly lower than those in cells incubated with STa toxin and serum sample from the negative control rabbit (16.8 pmole/ml). Similar results were observed when STa toxin was incubated with anti-fecal antibodies (FIG. 7a). The cGMP levels in cells treated with STa toxin and anti-'pLT₁₉₂:pSTa₁₂′ or anti-'pLT₁₉₂:pSTa₁₃′ fecal antibodies were 0.098 and 0.12 pmole/ml, respectively. That was similar compared to the cGMP level in cells treated with cell culture medium (0.13±0.03 pmole/ml), but differed significantly than the cGMP concentration in cells incubated with STa toxin and the fecal sample of the negative control rabbit (15.6 pmole/ml).

B. Human

Anti-STa and anti-LT antibodies in mouse serum, fecal suspension and intestinal washing samples (1:50 dilution) were titrated. Anti-STa antibodies were titrated in an STa ELISA by using STa ovalbumin-conjugate antigens, and anti-LT antibodies were titrated in a standard GM1 ELISA using CT as antigens. All samples were tested in triplicates, and HRP-conjugated goat-anti-mouse IgG and IgA (1:3300; Sigma) were used as the secondary antibodies. The cutoff OD values in ELISA were defined as the A405 background plus 0.4. The dilution that gave OD values above the cutoff was calculated for antibody titers that were expressed as the log 10 of the reciprocal dilution. (FIGS. 6b and 6c).

Anti-STa antibody titration showed that anti-STa IgG antibodies were detected in serum and fecal samples of the immunized mice, but anti-STa IgA antibodies were detected only in the fecal samples. Anti-STa IgG antibodies in sera of the mice immunized with fusion 1b, fusion 2b, fusion 3b, fusion 4b, and fusion 5b proteins were detected at titers (in log 10) of 1.68±0.18, 1.91±0.39, 1.49, 1.70±0.28, and 1.70±0.24, respectively (FIG. 6b). No anti-STa IgA antibodies were detected in the same serum samples. In contrast, anti-STa IgA antibodies in the fecal sample from the mice immunized with fusion 1b, fusion 2b, fusion 3b, fusion 4b, and fusion 5b proteins were detected at titers of 1.92±0.24, 1.93±0.04, 1.74±0.07, 1.99±0.27, and 2.21±0.10, respectively. Lower titers of anti-STa IgG antibodies were detected in the same fecal samples: 1.53±0.31, 1.52±0.01, 1.10±0.04, 1.56±0.27 and 1.90±0.21 (FIG. 6c).

Similarly, anti-LT IgG antibodies were detected in the serum samples, and anti-LT IgA and IgG in the fecal samples of the immunized mice (FIG. 7b). Serum anti-LT IgG antibodies were titrated at 2.24±0.23, 2.56±0.16, 1.64±0.15, 1.86±0.20 and 1.94±0.10 in the mice immunized with fusion 1b, 2b, 3b, 4b and 5b proteins, respectively. Lower anti-LT antibodies in fecal samples were detected. Anti-LT IgG titers were 1.53±0.12, 1.75±0.10, 0.54, 1.38±0.49 and 1.38±0.59; whereas the anti-LT IgA titers were 1.53±0.12, 1.75±0.14, 0.26±0.10, 1.16±0.27, and 1.11±0.50 in fecal samples of the mice immunized with fusion 1b, 2b, 3b, 4b and 5b proteins. No anti-LT and anti-STa antibodies were detected in serum or fecal samples of mice before immunization or the mice in the control group.

Neutralizing anti-STa antibodies against purified STa toxin were examined in T84-cells using a cGMP EIA kit (Assay Design, MI). Two ng STa toxin (diluted in 150-μl DMEM/F12 medium) was incubated with 150 μl of mouse serum or fecal suspension samples (1:5 dilution in DMEM/F12 medium, in triplicatess). After 1 hour incubation, 150 μl of the mixture was added to each well that contains 1−2×105 T84 cells, and incubated at 37° C. in 5% CO₂ for 2 hours. After washes, cells were lysed with 0.1M HCl (200 μl per well) and followed by a treatment with 0.1 M NaOH. Cell lysates were collected with a centrifugation at 660×g for 10 min at room temperature. Lysate supernatants were collected and tested for intracellular cGMP levels by following the manufacturer's protocols.

Elicited anti-STa antibodies neutralized STa toxin in vitro. After incubation with the fecal samples of the immunized mice, 2 ng STa toxin failed in increasing intracellular cGMP levels significantly in T-84 cells (FIG. 7b). The intracellular cGMP concentrations in cells treated with STa mixed with fecal samples (1:5 dilution) from the mice immunized with purified 6×His-tagged fusion 1b, fusion 2b, fusion 3b, fusion 4b, and fusion 5b proteins were 4.75±1.21, 4.77±0.65, 3.74±0.95, 2.80±1.25 and 3.30±0.73 μmol/ml, respectively. In contrast, the intracellular cGMP concentration in cells treated with 2 ng STa mixed with fecal samples of the control mice was 9.1±0.18 μmol/ml. The cGMP levels in cells incubated with STa and the fecal sample of the immunized mice were significantly lower than that in cells incubated with STa and fecal samples from the control mice.

### VIII. Immunization of a Pregnant Sow and Piglet Challenge Studies

Two pregnant sows from an isolated hog farm with no ETEC diarrhea outbreak were used in this study. Sow #1-4 was immunized with 0.5 mg purified porcine “LT₁₉₂:STa₁₃” fusion protein in an equal volume of Freund's complete adjuvant six-eight weeks before farrowing, and then followed by a boost injection with the same amount protein in incomplete adjuvant 4 weeks later. The other pregnant sow, #25-3, was not immunized and was used as a control. Serum samples from both sows were examined for preexisting anti-LT or anti-STa antibodies. Sows were transported and raised separately in an isolated and disinfected room a few days prior to farrowing. Colostrum samples were collected from each sow to test anti-LT and anti-STa antibody production. Two-day old suckling piglets were taken away from the mother momentarily, orally inoculated with 2×10⁹ CFUs overnight culture growth of the STa challenge strain 8823, and brought back to their mother. Piglets were observed every 4 hours for 72 hours. At the end of 72 hours, all piglets underwent necropsy and blood and small intestinal samples were collected for antigenicity and colonization studies.

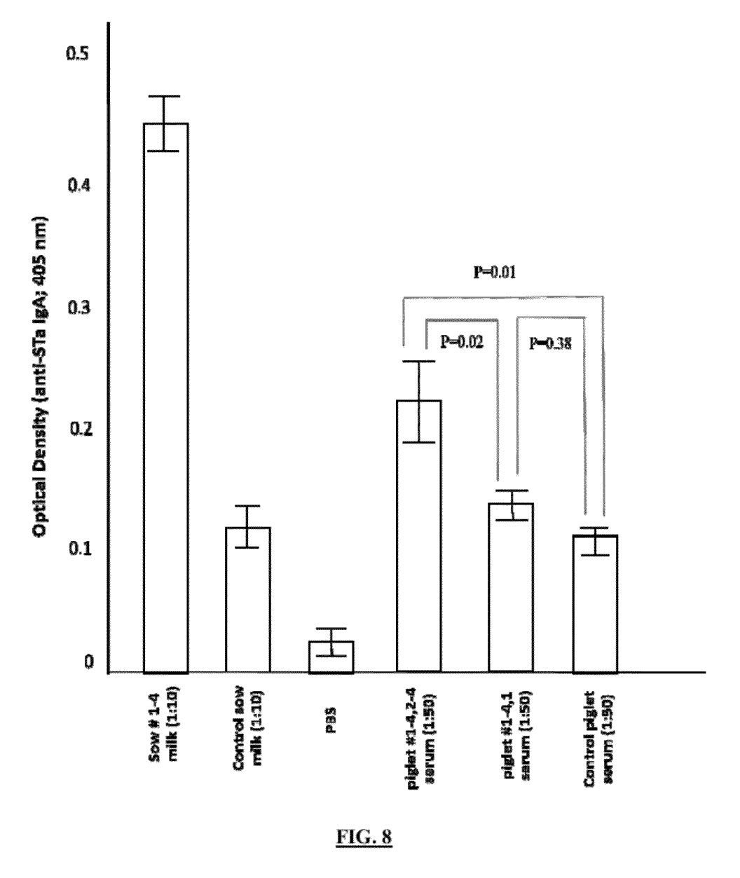

Suckling piglets born from the immunized sow were protected when challenged with a porcine STa ETEC strain. Four piglets were delivered from the immunized sow. After orally challenged with strain 8823, only one piglet showed mild diarrhea during the following 72 hours, whereas the remaining three piglets stayed healthy. Anti-STa IgA ELISA showed that colostrum (1:10 dilution) from the immunized sow had an OD value of 0.445±0.028, which was significantly different from the OD value in the colostrum from the negative control sow (0.122±0.017; p=0.01). Anti-STa IgA antibody was detected among three (of the four) piglets born by the immunized sow in an anti-STa IgA ELISA. Serum samples (1:50 dilution) from the three healthy piglets showed an OD value of 0.202±0.049, which is significantly different than the OD values from their diarrheal sibling (0.124±0.024; p=0.02) and the diarrheal piglets of the control (0.108±0.025; p=0.01) (FIG. 8).

Any aspect or design described herein as “exemplary” is not necessarily to be construed as preferred or advantageous over other aspects or designs. Exemplary embodiments may be implemented as a method or composition. The word “exemplary” is used herein to mean serving as an example, instance, or illustration.

All of the references cited herein are incorporated by reference in their entireties.

From the above discussion, one skilled in the art can ascertain the essential characteristics of the invention, and without departing from the spirit and scope thereof, can make various changes and modifications of the embodiments to adapt to various uses and conditions. Thus, various modifications of the embodiments, in addition to those shown and described herein, will be apparent to those skilled in the art from the foregoing description. Such modifications are also intended to fall within the scope of the appended claims.

What is claimed is:

1. An isolated polynucleotide comprising a coding sequence for an STa toxoid having at least 80% amino acid sequence identity to SEQ ID NO: 1 wherein the STa toxoid comprises at least one non-native amino acid, further wherein the non-native amino acid does not disrupt formation of a disulfide bond.

2. The isolated polynucleotide of claim 1 wherein at least one non-native amino acid is at amino acid 12, amino acid 13, or amino acid 14.

3. The isolated polynucleotide of claim 2 wherein the at least one non-native amino acid is at amino acid 13.

4. The isolated polynucleotide of claim 3 wherein the non-native amino acid is glutamine.

5. The isolated polynucleotide of claim 1 further comprising an isolated polynucleotide comprising a coding sequence for an LT polypeptide having at least 95% amino acid sequence identity to SEQ ID NO: 6, wherein the isolated polynucleotide comprising a coding sequence for the LT polypeptide is operably linked with the isolated polynucleotide comprising a coding sequence for the STa toxoid.

6. The isolated polynucleotide of claim 5 wherein the LT polypeptide is a toxoid.

7. The isolated polynucleotide of claim 6 wherein the LT toxoid comprises a non-native amino acid at amino acid 192.

8. The isolated polynucleotide of claim 5 wherein the isolated polynucleotide comprising a coding sequence for the LT polypeptide and the isolated polynucleotide comprising a coding sequence for the STa toxoid are operably linked through a linker.

9. The isolated polynucleotide of claim 8 wherein the linker is a glycine-proline-glycine-proline linker.

10. The isolated polynucleotide of any of claims 1-9 wherein the polypeptide comprises SEQ ID NO: 9.

11. An isolated polynucleotide comprising a coding sequence for an STa toxoid having at least 80% amino acid sequence identity to SEQ ID NO: 3, wherein the STa toxoid comprises at least one non-native amino acid, further wherein the non-native amino acid does not disrupt formation of a disulfide bond.

12. The isolated polynucleotide of claim 11 wherein at least one non-native amino acid is at amino acid amino acid 11, amino acid 12, or amino acid 13.

13. The isolated polynucleotide of claim 12 wherein the at least one non-native amino acid is at amino acid 13.

14. The isolated polynucleotide of claim 13 wherein the non-native amino acid is glutamine.

15. The isolated polynucleotide of claim 14 further comprising an isolated polynucleotide comprising a coding sequence for an LT polypeptide having at least 95% amino acid sequence identity to SEQ ID NO: 6, wherein the isolated polynucleotide comprising a coding sequence for the LT polypeptide is operably linked with the isolated polynucleotide comprising a coding sequence for the STa toxoid.

16. The isolated polynucleotide of claim 15 wherein the LT polypeptide is a toxoid.

17. The isolated polynucleotide of claim 16 wherein the LT toxoid comprises a non-native amino acid at amino acid 192.

18. The isolated polynucleotide of claim 17 wherein the isolated polynucleotide comprising a coding sequence for the LT polypeptide and the isolated polynucleotide comprising a coding sequence for the STa toxoid are operably linked through a linker.

19. The isolated polynucleotide of claim 18 wherein the linker is a glycine-proline-glycine-proline linker.

20. The isolated polynucleotide of any of claims 11-19 wherein the polypeptide comprises SEQ ID NO: 9.

21. A vector comprising at least one polynucleotide of any of claims 1-20.

22. A host cell transformed with at least one polynucleotide of any of claims 1-20.

23. A pharmaceutically effective therapeutic comprising the polynucleotide of any of claims 1-20.

24. The pharmaceutically effective therapeutic of claim 23 wherein the pharmaceutically effective therapeutic is a vaccine.

25. The vaccine of claim 24 wherein the vaccine comprises a host cell, further wherein the host cell produces the polypeptide of any of claims 1-20.

26. The vaccine of claim 25 wherein the host cell belongs to the group consisting of Escherichia coli strain 8474, 8475, 8552, and 8554.

27. A method of inducing an antigen specific immune response in a subject comprising administering an ETEC vaccine to a subject in need thereof, wherein the ETEC vaccine comprises an STa toxoid having at least 80% amino acid sequence identity to SEQ ID NO: 1, wherein the STa toxoid comprises at least one non-native amino acid, and yet further wherein the non-native amino acid does not disrupt formation of a disulfide bond.

28. The method of claim 27 wherein the ETEC vaccine comprises the vaccine of claim 24.

29. The method of claim 28 wherein the subject is a human.

30. The method of claim 29 wherein the human is an infant.

31. A method of inducing an antigen specific immune response in a subject comprising administering an ETEC vaccine to a subject, wherein the ETEC vaccine comprises an STa toxoid having at least 80% amino acid sequence identity to SEQ ID NO: 3, wherein the STa toxoid comprises at least one non-native amino acid, and yet further wherein the non-native amino acid does not disrupt formation of a disulfide bond.

32. The method of claim 31 wherein the subject is a porcine.

33. The method of claim 32 wherein the porcine is at risk of post-weaning diarrhea.

34. An isolated polynucleotide comprising
(a) a coding sequence for an STa toxoid having at least 80% amino acid sequence identity to SEQ ID NO: 3, wherein the STa toxoid comprises a glutamine at amino acid 13;
(b) a coding sequence for an LT toxoid having at least 95% amino acid sequence identity to SEQ ID NO: 6, wherein the LT toxoid comprises a glycine at amino acid 192; and
(c) a linker, wherein the linker operably links the isolated polynucleotide comprising a coding sequence for the STa toxoid and the isolated polynucleotide comprising a coding sequence for the LT toxoid.

35. A method of preventing accidental contamination of a food or water supply comprising administering an ETEC vaccine to a food or water supply, wherein the ETEC vaccine comprises an STa toxoid having at least 80% amino acid sequence identity to SEQ ID NO: 1, wherein the STa toxoid comprises at least one non-native amino acid, and yet further wherein the non-native amino acid does not disrupt formation of a disulfide bond

36. A method of preventing accidental contamination of a food or water supply comprising administering an ETEC vaccine to a food or water supply, wherein the ETEC vaccine comprises an STa toxoid having at least 80% amino acid sequence identity to SEQ ID NO: 3, wherein the STa toxoid comprises at least one non-native amino acid, and yet further wherein the non-native amino acid does not disrupt formation of a disulfide bond

37. A method of preventing accidental contamination of a food or water supply comprising administering an ETEC vaccine to a food or water supply, wherein the ETEC vaccine comprises an STa toxoid having at least 80% amino acid sequence identity to SEQ ID NO: 1, wherein the STa toxoid comprises at least one non-native amino acid, and yet further wherein the non-native amino acid does not disrupt formation of a disulfide bond.

38. A method of reducing the frequency of ETEC contamination of a food or water supply comprising administering an ETEC vaccine to a food or water supply, wherein the ETEC vaccine comprises an STa toxoid having at least 80% amino acid sequence identity to SEQ ID NO: 3, wherein the STa toxoid comprises at least one non-native amino acid, and yet further wherein the non-native amino acid does not disrupt formation of a disulfide bond


SEQUENCE LISTING (61 sequences)

SEQ ID NO: 1 - protein, 19 aa, E. coli
  misc_feature: human E. coli strain STa protein
NSSNYCCELCCNPACTGCY

SEQ ID NO: 2 - DNA, 63 bp, E. coli
  misc_feature: native human E. coli strain STa nucleotide sequence
atgaatagtagcaattactgctgtgaattgtgttgtaatcctgcttgtaccgggtgctattaa

SEQ ID NO: 3 - protein, 19 aa, E. coli
  misc_feature: native porcine E. coli strain STa protein
NNTFYCCELCCNPACAGCY

SEQ ID NO: 4 - DNA, 63 bp, E. coli
  misc_feature: native porcine E. coli strain STa nucleotide sequence
atgaacaacacattttactgctgtgaactttgttgtaatcctgcctgtgctggatgttattaa

SEQ ID NO: 5 - DNA, 868 bp, Artificial Sequence
  Synthetic DNA  Mutation of the eltAB gene
atgattgacatcatgttgcatataggttagataaaacaagtggttatctttccggattgtcttcttgtatgatatataagttttcctcgatgaaaaatataactttcattttttttattttattagcatcgccattatatgcaaatggcgacagattataccgtgctgactctagacccccagatgaaataaaacgtttccggagtcttatgcccagaggtaatgagtacttcgatagaggaactcaaatgaatattaatctttatgatcacgcgagaggaacacaaaccggctttgtcagatatgatgacggatatgtttccacttctcttagtttgagaagtgctcacttagcaggacagtatatattatcaggatattcacttactatatatatcgttatagcaaatatgtttaatgttaatgatgtaattagcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggagattcatcaagaacaatcacaggtgatacttgtaatgaggagacccagaatctgagcacaatatatctcagggaatatcaatcaaaagttaagaggcagatattttcagactatcagtcagaggttgacatatataacagaattcgggatgaattatgaataaagtaaaatgt

SEQ ID NO: 6 - protein, 382 aa, E. coli
  misc_feature: native LT protein
MKNITFIFFILLASPLYANGDRLYRADSRPPDEIKRSGGLMPRGHNEYFDRGTQMNINLYDHARGTQTGFVRYDDGYVSTSLSLRSAHLAGQYILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGDSSGTITGDTCNEETQNLSTIYLREYQSKVKRQIFSDYQSEVDIYNRIRDELMNKVKCYVLFTALLSSLYAHGAPQTITELCSEYRNTQIYTINDKILSYTESMAGKREMVIITFKSGETFQVGVPGSQHIDSQKKAIERMKDTLRITYLTETKIDKLCVWNNKTPNSIAAISMKN

SEQ ID NO: 7 - DNA, 12 bp, Artificial Sequence
  Synthetic oligonucleotide encoding a linker
gggccggggccc

SEQ ID NO: 8 - DNA, 21 bp, Artificial Sequence
  Synthetic oligonucleotide of an L-linker sequence
cgagctcggtacccggggatc

SEQ ID NO: 9 - protein, 402 aa, Artificial Sequence
  Synthetic fusion protein
MKNITFIFFILLASPLYANGDRLYRADSRPPDEIKRSGGLMPRGHNEYFDRGTQMNINLYDHARGTQTGFVRYDDGYVSTSLSLRSAHLAGQYILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGDSSGTITGDTCNEETQNLSTIYLREYQSKVKRQIFSDYQSEVDIYNRIRDELMNKVKCYVLFTALLSSLYAHGAPQTITELCSEYRNTQIYTINDKILSYTESMAGKREMVIITFKSGETFQVGVPGSQHIDSQKKAIERMKDTLRITYLTETKIDKLCVWNNKTPNSIAAISGPGPNNTYYCCELCCNPQCAGCY

SEQ ID NO: 10 - DNA, 21 bp, Artificial Sequence
  Synthetic oligonucleotide  184EcoRV-F
gtcaggcaccgtgtatgaaat

SEQ ID NO: 11 - DNA, 20 bp, Artificial Sequence
  Synthetic oligonucleotide  pBREagI-R
gtccctgatggtcgtcatct

SEQ ID NO: 12 - DNA, 19 bp, Artificial Sequence
  Synthetic oligonucleotide  STaSfcI-F
gtgaaacaacctgtaggga

SEQ ID NO: 13 - DNA, 18 bp, Artificial Sequence
  Synthetic oligonucleotide  STaEagI-R
gtggagccggccgaaaca

SEQ ID NO: 14 - DNA, 24 bp, Artificial Sequence
  Synthetic oligonucleotide  STaHindIII-F
tgcaaaataagcttaactaatctc

SEQ ID NO: 15 - DNA, 19 bp, Artificial Sequence
  Synthetic oligonucleotide  STaBamH1-R
gtggagccggatccaacag

SEQ ID NO: 16 - DNA, 24 bp, Artificial Sequence
  Synthetic oligonucleotide  pSTa11K-F
gaactttgttgtaaacctgcctgt

SEQ ID NO: 17 - DNA, 24 bp, Artificial Sequence
  Synthetic oligonucleotide  pSTa11K-R
acaggcaggtttacaacaaagttc

SEQ ID NO: 18 - DNA, 24 bp, Artificial Sequence
  Synthetic oligonucleotide  pSTa12F-F
ctttgttgtaattttgcctgtgcc

SEQ ID NO: 19 - DNA, 24 bp, Artificial Sequence
  Synthetic oligonucleotide  pSTa12F-R
ggcacaggcaaaattacaacaaag

SEQ ID NO: 20 - DNA, 24 bp, Artificial Sequence
  Synthetic oligonucleotide  pSTa13Q-F
tgttgtaatcctcagtgtgctgga

SEQ ID NO: 21 - DNA, 24 bp, Artificial Sequence
  Synthetic oligonucleotide  pSTa13Q-R
tccagcacactgaggattacaaca

SEQ ID NO: 22 - DNA, 41 bp, Artificial Sequence
  Synthetic oligonucleotide  pLTSTa-F
gcaatcagtgggccggggcccatgaacaacttttactgctg

SEQ ID NO: 23 - DNA, 44 bp, Artificial Sequence
  Synthetic oligonucleotide  pLTSTa-R
gttgttcatgggccccggcccactgattgccgcaattgaattgg

SEQ ID NO: 24 - DNA, 24 bp, Artificial Sequence
  Synthetic oligonucleotide  STa12NS-R
ataacatccagcacaggcaaaatt

SEQ ID NO: 25 - DNA, 21 bp, Artificial Sequence
  Synthetic oligonucleotide  ST13NS-R
ataacatccagcacactgagg

SEQ ID NO: 26 - DNA, 26 bp, Artificial Sequence
  Synthetic oligonucleotide  LT-R
gtttttcatactgattgccccaattg

SEQ ID NO: 27 - DNA, 22 bp, Artificial Sequence
  Synthetic oligonucleotide  STa-F
aacaacacattttactgctgtg

SEQ ID NO: 28 - DNA, 26 bp, Artificial Sequence
  Synthetic oligonucleotide  hLT-F
atgattgacatcatgttgcatatagg

SEQ ID NO: 29 - DNA, 19 bp, Artificial Sequence
  Synthetic oligonucleotide  STaNheI-F
cgacgtgtttgctagctaa

SEQ ID NO: 30 - DNA, 21 bp, Artificial Sequence
  Synthetic oligonucleotide  LTNheI-F
atcctcgctagcatgttttat

SEQ ID NO: 31 - DNA, 34 bp, Artificial Sequence
  Synthetic oligonucleotide  LTEagI-R
gcgtcggccgctactagttttccatactgattgc

SEQ ID NO: 32 - DNA, 27 bp, Artificial Sequence
  Synthetic oligonucleotide  pBRNheI-F
tgctaacgcagtcaggcaccgtgtatg

SEQ ID NO: 33 - DNA, 23 bp, Artificial Sequence
  Synthetic oligonucleotide  LT192-F
aattcatcaggaacaattacagg

SEQ ID NO: 34 - DNA, 23 bp, Artificial Sequence
  Synthetic oligonucleotide  LT192-R
cctgtaattgttcctgatgaatt

SEQ ID NO: 35 - DNA, 24 bp, Artificial Sequence
  Synthetic oligonucleotide  STa13-F
ttgtgttgtaattttgcttgtacc

SEQ ID NO: 36 - DNA, 24 bp, Artificial Sequence
  Synthetic oligonucleotide  STa13-R
ggtacaagcaaaattacaacacaa

SEQ ID NO: 37 - DNA, 42 bp, Artificial Sequence
  Synthetic oligonucleotide  STa13LT192-F
gcaatcagtgggccggggcccatgaatagtagcaattactgc

SEQ ID NO: 38 - DNA, 44 bp, Artificial Sequence
  Synthetic oligonucleotide  LT192STa13-R
actattcatgggccccggcccactgattgccgcaattgaattgg

SEQ ID NO: 39 - DNA, 45 bp, Artificial Sequence
  Synthetic oligonucleotide  STa13-L-LT192-F
agtgatccccgggtaccgagctcgatgaatagtagcaattactgc

SEQ ID NO: 40 - DNA, 47 bp, Artificial Sequence
  Synthetic oligonucleotide  STa13-L-LT192-R
catcgagctcggtacccggggatcactgattgccgcaattgaattgg

SEQ ID NO: 41 - DNA, 45 bp, Artificial Sequence
  Synthetic oligonucleotide  STa13LT192-F
accgggtgctatgggccggggcccaatggcgacaaattataccgt

SEQ ID NO: 42 - DNA, 45 bp, Artificial Sequence
  Synthetic oligonucleotide  LT192STa13-R
gtcgccattgggccccggcccatagcacccggtacaagcaaaatt

SEQ ID NO: 43 - DNA, 46 bp, Artificial Sequence
  Synthetic oligonucleotide  LT192A1STa13-F
tcatcaggagggccggtcgacatgaatagtagcaattactgctgtg

SEQ ID NO: 44 - DNA, 48 bp, Artificial Sequence
  Synthetic oligonucleotide  STa13LT192A1-R
gctactattcatgtcgaccggccctcctgatgaatttccacaaccttg

SEQ ID NO: 45 - DNA, 34 bp, Artificial Sequence
  Synthetic oligonucleotide  STa13LT192A2-F
gggtgctatacaattacaggtgatacttgtaatg

SEQ ID NO: 46 - DNA, 39 bp, Artificial Sequence
  Synthetic oligonucleotide  LT192A2STa13-R
cacctgtaattgtatagcacccggtacaagcaaaattac

SEQ ID NO: 47 - DNA, 30 bp, Artificial Sequence
  Synthetic oligonucleotide  LT192BSSTa13-F
gcatacggaatgaatagtagcaattactgc

SEQ ID NO: 48 - DNA, 29 bp, Artificial Sequence
  Synthetic oligonucleotide  STa13LT192B-R
actattcattccgtatgcacatagagagg

SEQ ID NO: 49 - DNA, 47 bp, Artificial Sequence
  Synthetic oligonucleotide  STa13LT192B-F
gggtgctatgggccggggcccgctccccagtctattacagaactatg

SEQ ID NO: 50 - DNA, 47 bp, Artificial Sequence
  Synthetic oligonucleotide  LT192BSTa13-R
ctggggagcgggccccggcccatagcacccggtacaagcaaaattac

SEQ ID NO: 51 - DNA, 28 bp, Artificial Sequence
  Synthetic oligonucleotide  LT192NheI-F3
gtttgctagcaatggcgacaaattatac

SEQ ID NO: 52 - DNA, 34 bp, Artificial Sequence
  Synthetic oligonucleotide  STa13EagI-R1
gcgacggccgttattaatagcacccggtacaagc

SEQ ID NO: 53 - DNA, 27 bp, Artificial Sequence
  Synthetic oligonucleotide  STa13NheI-F2
gtagctagcatgaatagtagcaattac

SEQ ID NO: 54 - DNA, 28 bp, Artificial Sequence
  Synthetic oligonucleotide  LT192BamHI-R1
gcgtggatccctactagttttccatact

SEQ ID NO: 55 - DNA, 46 bp, Artificial Sequence
  Synthetic oligonucleotide  LT192A-LL-LT192B-F
gaattagatccccgggtaccgagctcggctccccagtctattacag

SEQ ID NO: 56 - DNA, 49 bp, Artificial Sequence
  Synthetic oligonucleotide  LT192A-LL-LT192B-R
ctggggagccgagctcggtacccggggatctaattcattccgaattctg

SEQ ID NO: 57 - DNA, 37 bp, Artificial Sequence
  Synthetic oligonucleotide  LT192A-SL-LT192B-F
gaattagggccggggcccgctccccagtctattacag

SEQ ID NO: 58 - DNA, 40 bp, Artificial Sequence
  Synthetic oligonucleotide  LT192A-SL-LT192B-R
ctggggagcgggccccggccctaattcattccgaattctg

SEQ ID NO: 59 - DNA, 45 bp, Artificial Sequence
  Synthetic oligonucleotide  LT192A-LL-STa13-F
gaattagatccccgggtaccgagctcgatgaatagtagcaattac

SEQ ID NO: 60 - DNA, 49 bp, Artificial Sequence
  Synthetic oligonucleotide  LT192A-LL-STa13-R
actattcatcgagctcggtacccggggatctaattcattccgaattctg

SEQ ID NO: 61 - protein, 4 aa, Artificial Sequence
  Synthetic peptide
GPGP